SEQUENCE LISTING (33 sequences)

SEQ ID NO: 1 - protein, 248 aa, Homo sapiens
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 2 - protein, 228 aa, Homo sapiens
ETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 3 - protein, 141 aa, Homo sapiens
AASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 4 - protein, 115 aa, Homo sapiens
VGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 5 - protein, 148 aa, Homo sapiens
PDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 6 - protein, 381 aa, Artificial Sequence
  Recombinantly-produced fusion protein
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEINITFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 7 - protein, 383 aa, Artificial Sequence
  Recombinantly-produced fusion protein
AKTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 8 - protein, 352 aa, Artificial Sequence
  Recombinantly-produced fusion protein
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGATSKQVGNKFFLTNGEEVITFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 9 - protein, 383 aa, Artificial Sequence
  Recombinantly-produced fusion protein
AKTEPKSSDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 10 - protein, 10 aa, Homo sapiens
NEGEPNNAGS

SEQ ID NO: 11 - protein, 15 aa, Homo sapiens
LLLKNGQWNDVPCST

SEQ ID NO: 12 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 13 - protein, 278 aa, Homo sapiens [sequence omitted]

SEQ ID NO: 14 - protein, 375 aa, Homo sapiens
MLLFLLSALVLLTQPLGYLEAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGALGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 15 - protein, 277 aa, Homo sapiens
MNGFASLLRRNQFILLVLFLLQIQSLGLDIDSRPTAEVCATHTISPGPKGDDGEKGDPGEEGKHGKVGRMGPKGIKGELGDMGDQGNIGKTGPIGKKGDKGEKGLLGIPGEKGKAGTVCDCGRYRKFVGQLDISIARLKTSMKFVKNVIAGIRETEEKFYYIVQEEKNYRESLTHCRIRGGMLAMPKDEAANTLIADYVAKSGFFRVFIGVNDLEREGQYMFTDNTPLQNYSNWNEGEPSDPYGHEDCVEMLSSGRWNDTECHLTMYFVCEFIKKKK

SEQ ID NO: 16 - protein, 742 aa, Homo sapiens
MKDDFAEEEEVQSFGYKRFGIQEGTQCTKCKNNWALKFSIILLYILCALLTITVAILGYKVVEKMDNVTGGMETSRQTYDDKLTAVESDLKKLGDQTGKKAISTNSELSTFRSDILDLRQQLREITEKTSKNKDTLEKLQASGDALVDRQSQLKETLENNSFLITTVNKTLQAYNGYVTNLQQDTSVLQGNLQNQMYSHNVVIMNLNNLNLTQVQQRNLITNLQRSVDDTSQAIQRIKNDFQNLQQVFLQAKKDTDWLKEKVQSLQTLAANNSALAKANNDTLEDMNSQLNSFTGQMENITTISQANEQNLKDLQDLHKDAENRTAIKFNQLEERFQLFETDIVNIISNISYTAHHLRTLTSNLNEVRTTCTDTLTKHTDDLTSLNNTLANIRLDSVSLRMQQDLMRSRLDTEVANLSVIMEEMKLVDSKHGQLIKNFTILQGPPGPRGPRGDRGSQGPPGPTGNKGQKGEKGEPGPPGPAGERGPIGPAGPPGERGGKGSKGSQGPKGSRGSPGKPGPQGPSGDPGPPGPPGKEGLPGPQGPPGFQGLQGTVGEPGVPGPRGLPGLPGVPGMPGPKGPPGPPGPSGAVVPLALQNEPTPAPEDNGCPPHWKNFTDKCYYFSVEKEIFEDAKLFCEDKSSHLVFINTREEQQWIKKQMVGRESHWIGLTDSERENEWKWLDGTSPDYKNWKAGQPDNWGHGHGPGEDCAGLIYAGQWNDFQCEDVNNFICEKDRETVLSSAL

SEQ ID NO: 17 - protein, 321 aa, Bos taurus
MLPLPLSILLLLTQSQSFLGEEMDVYSEKTLTDPCTVVVCAPPADSLRGHDGRDGKEGPQGEKGDPGPPGMPGPAGREGPSGRQGSMGPPGTPGPKGEPGPEGGVGAPGMPGSPGPTGLKGERGTPGPGGAIGPQGPSGAMGPPGLKGDRGDPGEKGAKGETSVLEVDTLRQRMRNLEGEVQRLQNIVTQYRKAVLFPDGQAVGEKIFKTAGAVKSYSDAEQLCREAKGQLASPRSSAENEAVTQLVRAKNKHAYLSMNDISKEGKFTYPTGGSLDYSNWAPGEPNNRAKDEGPENCLEIYSDGNWNDIECREERLVICEF

SEQ ID NO: 18 - protein, 371 aa, Bos taurus
MLLLPLSVLLLLTQPWRSLGAEMKIYSQKTLANGCTLVVCRPPEGGLPGRDGQDGREGPQGEKGDPGSPGPAGRAGRPGPAGPIGPKGDNGSAGEPGPKGDTGPPGPPGMPGPAGREGPSGKQGSMGPPGTPGPKGDTGPKGGMGAPGMQGSPGPAGLKGERGAPGELGAPGSAGVAGPAGAIGPQGPSGARGPPGLKGDRGDPGERGAKGESGLADVNALKQRVTILEGQLQRLQNAFSRYKKAVLFPDGQAVGKKIFKTAGAVKSYSDAQQLCREAKGQLASPRSAAENEAVAQLVRAKNNDAFLSMNDISTEGKFTYPTGESLVYSNWASGEPNNNNAGQPENCVQIYREGKWNDVPCSEPLLVICEF

SEQ ID NO: 19 - protein, 271 aa, Homo sapiens
MRGNLALVGVLISLAFLSLLPSGHPQPAGDDACSVQILVPGLKGDAGEKGDKGAPGRPGRVGPTGEKGDMGDKGQKGSVGRHGKIGPIGSKGEKGDSGDIGPPGPNGEPGLPCECSQLRKAIGEMDNQVSQLTSELKFIKNAVAGVRETESKIYLLVKEEKRYADAQLSCQGRGGTLSMPKDEAANGLMAAYLAQAGLARVFIGINDLEKEGAFVYSDHSPMRTFNKWRSGEPNNAYDEEDCVEMVASGGWNDVACHTTMYFMCEFDKENM

SEQ ID NO: 20 - protein, 371 aa, Bos taurus
MLLLPLSVLLLLTQPWRSLGAEMTTFSQKILANACTLVMCSPLESGLPGHDGQDGRECPHGEKGDPGSPGPAGRAGRPGWVGPIGPKGDNGFVGEPGPKGDTGPRGPPGMPGPAGREGPSGKQGSMGPPGTPGPKGETGPKGGVGAPGIQGFPGPSGLKGEKGAPGETGAPGRAGVTGPSGAIGPQGPSGARGPPGLKGDRGDPGETGAKGESGLAEVNALKQRVTILDGHLRRFQNAFSQYKKAVLFPDGQAVGEKIFKTAGAVKSYSDAEQLCREAKGQLASPRSSAENEAVTQMVRAQEKNAYLSMNDISTEGRFTYPTGEILVYSNWADGEPNNSDEGQPENCVEIFPDGKWNDVPCSKQLLVICEF

SEQ ID NO: 21 - protein, 326 aa, Homo sapiens
MELSGATMARGLAVLLVLFLHIKNLPAQAADTCPEVKVVGLEGSDKLTILRGCPGLPGAPGPKGEAGVIGERGERGLPGAPGKAGPVGPKGDRGEKGMRGEKGDAGQSQSCATGPRNCKDLLDRGYFLSGWHTIYLPDCRPLTVLCDMDTDGGGWTVFQRRMDGSVDFYRDWAAYKQGFGSQLGEFWLGNDNIHALTAQGSSELRVDLVDFEGNHQFAKYKSFKVADEAEKYKLVLGAFVGGSAGNSLTGHNNNFFSTKDQDNDVSSSNCAEKFQGAWWYADCHASNLNGLYLMGPHESYANGINWSAAKGYKYSYKVSEMKVRPA

SEQ ID NO: 22 - protein, 313 aa, Homo sapiens
MELDRAVGVLGAATLLLSFLGMAWALQAADTCPEVKMVGLEGSDKLTILRGCPGLPGAPGPKGEAGTNGKRGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 23 - protein, 299 aa, Homo sapiens
MDLLWILPSLWLLLLGGPACLKTQEHPSCPGPRELEASKVVLLPSCPGAPGSPGEKGAPGPQGPPGPPGKMGPKGEPGDPVNLLRCQEGPRNCRELLSQGATLSGWYHLCLPEGRALPVFCDMDTEGGGWLVFQRRQDGSVDFFRSWSSYRAGFGNQESEFWLGNENLHQLTLQGNWELRVELEDFNGNRTFAHYATFRLLGEVDHYQLALGKFSEGTAGDSLSLHSGRPFTTYDADHDSSNSNCAVIVHGAWWYASCYRSNLNGRYAVSEAAAHKYGIDWASGRGVGHPYRRVRMMLR

SEQ ID NO: 24 - protein, 786 aa, Homo sapiens
MTSIFHFAIIFMLILQIRIQLSEESEFLVDRSKNGLIHVPKDLSQKTTILNISQNYISELWTSDILSLSKLRILIISHNRIQYLDISVFKFNQELEYLDLSHNKLVKISCHPTVNLKHLDLSFNAFDALPICKEFGNMSQLKFLGLSTTHLEKSSVLPIAHLNISKVLLVLGETYGEKEDPEGLQDFNTESLHIVFPTNKEFHFILDVSVKTVANLELSNIKCVLEDNKCSYFLSILAKLQTNPKLSNLTLNNIETTWNSFIRILQLVWHTTVWYFSISNVKLQGQLDFRDFDYSGTSLKALSIHQVVSDVFGFPQSYIYEIFSNMNIKNFTVSGTRMVHMLCPSKISPFLHLDFSNNLLTDTVFENCGHLTELETLILQMNQLKELSKIAEMTTQMKSLQQLDISQNSVSYDEKKGDCSWTKSLLSLNMSSNILTDTIFRCLPPRIKVLDLHSNKIKSIPKQVVKLEALQELNVAFNSLTDLPGCGSFSSLSVLIIDHNSVSHPSADFFQSCQKMRSIKAGDNPFQCTCELGEFVKNIDQVSSEVLEGWPDSYKCDYPESYRGTLLKDFHMSELSCNITLLIVTIVATMLVLAVTVTSLCSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 25 - protein, 784 aa, Homo sapiens
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 26 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 27 - protein, 839 aa, Homo sapiens
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 28 - protein, 858 aa, Homo sapiens
MGDHLDLLLGVVLMAGPVFGIPSCSFDGRIAFYRFCNLTQVPQVLNTTERLLLSFNYIRTVTASSFPFLEQLQLLELGSQYTPLTIDKEAFRNLPNLRILDLGSSKIYFLHPDAFQGLFHLFELRLYFCGLSDAVLKDGYFRNLKALTRLDLSKNQIRSLYLHPSFGKLNSLKSIDFSSNQIFLVCEHELEPLQGKTLSFFSLAANSLYSRVSVDWGKCMNPFRNMVLEILDVSGNGWTVDITGNFSNAISKSQAFSLILAHHIMGAGFGFHNIKDPDQNTFAGLARSSVRHLDLSHGFVFSLNSRVFETLKDLKVLNLAYNKINKIADEAFYGLDNLQVLNLSYNLLGELYSSNFYGLPKVAYIDLQKNHIAIIQDQTFKFLEKLQTLDLRDNALTTIHFIPSIPDIFLSGNKLVTLPKINLTANLIHLSENRLENLDILYFLLRVPHLQILILNQNRFSSCSGDQTPSENPSLEQLFLGENMLQLAWETELCWDVFEGLSHLQVLYLNHNYLNSLPPGVFSHLTALRGLSLNSNRLTVLSHNDLPANLEILDISRNQLLAPNPDVFVSLSVLDITHNKFICECELSTFINWLNHTNVTIAGPPADIYCVYPDSFSGVSLFSLSTEGCDEEEVLKSLKFSLFIVCTVTLTLFLMTILTVTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDLQDVGWFLHKLSQQILKKEKEKKKDNNIPLQTVATIS

SEQ ID NO: 29 - protein, 796 aa, Homo sapiens
MTKDKEPIVKSFHFVCLMIIIVGTRIQFSDGNEFAVDKSKRGLIHVPKDLPLKTKVLDMSQNYIAELQVSDMSFLSELTVLRLSHNRIQLLDLSVFKFNQDLEYLDLSHNQLQKISCHPIVSFRHLDLSFNDFKALPICKEFGNLSQLNFLGLSAMKLQKLDLLPIAHLHLSYILLDLRNYYIKENETESLQILNAKTLHLVFHPTSLFAIQVNISVNTLGCLQLTNIKLNDDNCQVFIKFLSELTRGSTLLNFTLNHIETTWKCLVRVFQFLWPKPVEYLNIYNLTIIESIREEDFTYSKTTLKALTIEHITNQVFLFSQTALYTVFSEMNIMMLTISDTPFIHMLCPHAPSTFKFLNFTQNVFTDSIFEKCSTLVKLETLILQKNGLKDLFKVGLMTKDMPSLEILDVSWNSLESGRHKENCTWVESIVVLNLSSNMLTDSVFRCLPPRIKVLDLHSNKIKSVPKQVVKLEALQELNVAFNSLTDLPGCGSFSSLSVLIIDHNSVSHPSADFFQSCQKMRSIKAGDNPFQCTCELREFVKNIDQVSSEVLEGWPDSYKCDYPESYRGSPLKDFHMSELSCNITLLIVTIGATMLVLAVTVTSLCIYLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 30 - protein, 1049 aa, Homo sapiens
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 31 - protein, 1041 aa, Homo sapiens
MENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 32 - protein, 1032 aa, Homo sapiens
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 33 - protein, 811 aa, Homo sapiens
MRLIRNIYIFCSIVMTAEGDAPELPEERELMTNCSNMSLRKVPADLTPATTTLDLSYNLLFQLQSSDFHSVSKLRVLILCHNRIQQLDLKTFEFNKELRYLDLSNNRLKSVTWYLLAGLRYLDLSFNDFDTMPICEEAGNMSHLEILGLSGAKIQKSDFQKIAHLHLNTVFLGFRTLPHYEEGSLPILNTTKLHIVLPMDTNFWVLLRDGIKTSKILEMTNIDGKSQFVSYEMQRNLSLENAKTSVLLLNKVDLLWDDLFLILQFVWHTSVEHFQIRNVTFGGKAYLDHNSFDYSNTVMRTIKLEHVHFRVFYIQQDKIYLLLTKMDIENLTISNAQMPHMLFPNYPTKFQYLNFANNILTDELFKRTIQLPHLKTLILNGNKLETLSLVSCFANNTPLEHLDLSQNLLQHKNDENCSWPETVVNMNLSYNKLSDSVFRCLPKSIQILDLNNNQIQTVPKETIHLMALRELNIAFNFLTDLPGCSHFSRLSVLNIEMNFILSPSLDFVQSCQEVKTLNAGRNPFRCTCELKNFIQLETYSEVMMVGWSDSYTCEYPLNLRGTRLKDVHLHELSCNTALLIVTIVVIMLVLGLAVAFCCLHFDLPWYLRMLGQCTQTWHRVRKTTQEQLKRNVRFHAFISYSEHDSLWVKNELIPNLEKEDGSILICLYESYFDPGKSISENIVSFIEKSYKSIFVLSPNFVQNEWCHYEFYFAHHNLFHENSDHIILILLEPIPFYCIPTRYHKLKALLEKKAYLEWPKDRRKCGLFWANLRAAINVNVLATREMYELQTFTELNEESRGSTISLMRTDCL

(19) United States
(12) Patent Application Publication
(10) Pub. No.: US 2024/0199719 A1
(43) Pub. Date: Jun. 20, 2024

(54) THERAPEUTIC COMPOSITIONS COMPRISING MICROBE-TARGETING MOLECULES AND THEIR USE IN METHODS OF PREVENTING AND TREATING INFECTIOUS DISEASE

(71) Applicant: MIRAKI INNOVATION THINK TANK LLC, Cambridge, MA (US)

(72) Inventors: Christopher VELIS, Lexington, MA (US); Keith CRAWFORD, Westwood, MA (US)

(73) Assignee: MIRAKI INNOVATION THINK TANK LLC, Cambridge, MA (US)

(21) Appl. No.: 18/038,541

(22) PCT Filed: Aug. 20, 2021
(86) PCT No.: PCT/US21/46895

§ 371 Date: May 24, 2023

Related U.S. Application Data
(60) Provisional application No. 63/169,603, filed on Apr. 1, 2021.
(60) Provisional application No. 63/071,611, filed on Aug. 28, 2020.

(51) Int. Cl. C07K 14/705; A61K 38/00; A61K 45/06; C07K 14/47; C07K 14/75; C07K 14/78

(52) U.S. Cl. CPC C07K 14/70503; A61K 45/06; C07K 14/47; C07K 14/705; C07K 14/75; C07K 14/78; A61K 38/00; C07K 2319/02; C07K 2319/03; C07K 2319/30

(57) ABSTRACT

The present invention provides compositions comprising microbe-targeting molecules (MTMs) that bind microbe-associated molecular patterns, and methods of using the compositions in the prevention and treatment of infectious disease.

### SEQUENCE LISTING

A sequence listing in electronic (ASCII text file) format is filed with this application and incorporated herein by reference. The name of the ASCII text file is “2021_1399A_ST25.txt”; the file was created on Aug. 17, 2021; the size of the file is 132 KB.

### FIELD OF THE INVENTION

The technology described herein generally relates to compositions comprising biological molecules that can bind to microbes and microbe components in a sample or at a target area, including bodily fluids (e.g. blood and tissues); locations on or within a body, such as the human body; food; water; and environmental surfaces.

### BACKGROUND

Infectious diseases are caused by pathogenic microorganisms (e.g., bacteria, virus, parasites, or fungi) which can directly damage tissue, and resulting inflammatory responses can cause further damage and lead to septic shock and death. Sepsis is a major cause of death in American intensive care units. Methods for prevention, early intervention and treatment of infectious disease are needed.

### BRIEF SUMMARY

The present invention is directed to compositions comprising microbe-targeting molecules (MTMs) and/or engineered MTMs that have the shared characteristic of binding to one or more microbe-associated molecular patterns (MAMPs), and to the use of such compositions in the treatment and/or prevention of infectious disease.

The important basis of the MTMs used in the compositions of the invention, and the related methods, is that these constructs contact and bind microbes and microbial components based on the identity of the MAMP produced by the microbe, rather than the identity of microbe itself. While some MAMPs are produced by only a single species of microbe, other MAMPs are shared across species. Thus, while some MTMs of the invention bind to only MAMPs of a particular species of microbe, other MTMs of the invention can bind to MAMPs produced by all members of a particular class, order, family, genus or sub-genus of microbe.

As used herein, “MTM” and “engineered MTM” refers to any of the molecules described herein (or described in patents or patent application incorporated by reference) that can bind to a microbe or microbe component. Unless the context indicates otherwise, the term “MTM” is used to describe all MTMs of the invention, both naturally-occurring and engineered forms of these constructs.

Given that the MTMs of the invention are defined based on their binding activity, it will be apparent that both naturally-occurring and engineered MTMs will comprise at least one microbe-binding domain, i.e. a domain that recognizes and binds to one or more MAMPs (including, at least two, at least three, at least four, at least five, or more) as described herein. A microbe-binding domain can be a naturally-occurring or a synthetic molecule. In some aspects, a microbe-binding domain can be a recombinant molecule. In addition to the microbe-binding domain, the MTMs of the invention will typically have one or more additional domains that may include, but are not limited to, an oligomerization domain, a signal domain, an anchor domain, a collagen-like domain, a fibrinogen-like domain, an immunoglobulin domain, and an immunoglobulin-like domain.

As non-limiting examples of the MTMs of the invention, three broad categories of suitable MTMs are encompassed within the invention, namely: (i) collectin-based MTMs, (ii) ficolin-based MTMs, and (iii) toll-like receptor-based MTMs.

Thus, and in a first embodiment, the present invention is directed to compositions comprising collectin-based MTMs. The collectin-based MTMs are either naturally-occurring collectin proteins or engineered MTM fusion proteins that comprise at least one collectin microbe-binding domain and at least one additional domain.

The naturally-occurring collectin protein may be any one of (i) mannose-binding lectin (MBL), (ii) surfactant protein A (SP-A), (iii) surfactant protein D (SP-D), (iv) collectin liver 1 (CL-L1), (v) collectin placenta 1 (CL-P1), (vi) conglutinin collectin of 43 kDa (CL-43), (vii) collectin of 46 kDa (CL-46), (viii) collectin kidney 1 (CL-K1), (ix) conglutinin, and (x) a sequence variant having at least 85% sequence identity to any one of (i)-(ix).

In one aspect of this embodiment, the collectin is (i) a naturally-occurring MBL, (ii) a truncated form of naturally-occurring MBL, (iii) an engineered form of MBL, or (iv) a sequence variant having at least 85% sequence identity to any one of (i), (ii) or (iii).

In a specific aspect of this embodiment, the collectin is a naturally-occurring MBL as set forth in SEQ ID NO:1 or a sequence variant having at least 85% sequence identity with SEQ ID NO:1 that retains the activity of the native protein.

In another specific aspect of this embodiment, the collectin is a truncated form of naturally-occurring MBL as set forth in any one of SEQ ID NOs:2-5 or a sequence variant having at least 85% sequence identity with any one of SEQ ID NOs:2-5 that retains the activity of the native protein.

The collectin microbe-binding domain of the collectin-based engineered MTMs comprises a carbohydrate recognition domain (CRD) of a collectin. The collectin may be any one of (i) mannose-binding lectin (MBL), (ii) surfactant protein A (SP-A), (iii) surfactant protein D (SP-D), (iv) collectin liver 1 (CL-L1), (v) collectin placenta 1 (CL-P1), (vi) conglutinin collectin of 43 kDa (CL-43), (vii) collectin of 46 kDa (CL-46), (viii) collectin kidney 1 (CL-K1), (ix) conglutinin, and (x) a sequence variant having at least 85% sequence identity to any one of (i)-(ix).

The at least one additional domain of the collectin-based engineered MTMs may be one or more of (xi) a collectin cysteine-rich domain, (xii) a collectin collagen-like domain, (xiii) a collectin coiled-coil neck domain, (xiv) a ficolin short N-terminal domain, (xv) a ficolin collagen-like domain, (xvi) a Toll-like receptor (TLR) transmembrane helix, (xvii) a TLR C-terminal cytoplasmic signaling domain, (xviii) an oligomerization domain, (xix) a signal domain, (xx) an anchor domain, (xxi) a collagen-like domain, (xxii) a fibrinogen-like domain, (xxiii) an immunoglobulin domain, (xxiv) an immunoglobulin-like domain, and (xxv) a sequence variant having at least 85% sequence identity to any one of (xi)-(xxiv).

In certain aspects of this embodiment, the at least one additional domain is an immunoglobulin domain. For example, the immunoglobulin domain may comprise the amino acid sequence of SEQ ID NO:12 or a sequence variant thereof having at least 85% sequence identity to SEQ ID NO:12.

In certain aspects of this embodiment, the collectin microbe-binding domain comprises the CRD of MBL or a sequence variant thereof having at least 85% sequence identity to the CRD of MBL. For example, the CRD of MBL may comprise the amino acid sequence of any one of SEQ ID NOs:1, 2, 3, 4, and 5 or a sequence variant thereof having at least 85% sequence identity to any one of SEQ ID NOs:1, 2, 3, 4, and 5.

In certain aspects of this embodiment, the MTM comprises the CRD of MBL or a sequence variant thereof having at least 85% sequence identity to the CRD of MBL and an immunoglobulin domain. For example, the CRD of MBL may comprise the amino acid sequence of any one of SEQ ID NOs:1, 2, 3, 4, and 5 or a sequence variant thereof having at least 85% sequence identity to any one of SEQ ID NOs:1, 2, 3, 4, and 5, and an immunoglobulin domain comprising the amino acid sequence of SEQ ID NO12: or a sequence variant thereof having at least 85% sequence identity to SEQ ID NO:12.

In certain aspects of this embodiment, the compositions comprise at least one collectin-based engineered MTM, wherein the collectin-based engineered MTM is an FcMBL of SEQ ID NO:6, 7, 8 or 9, or a sequence variant thereof having at least 85% sequence identity to SEQ ID NO:6, 7, 8 or 9. FcMBL MTMs comprise a mannose-binding ligand (MBL) linked to the Fc domain of human IgG (Fc).

In certain other aspects of this embodiment, the compositions comprise at least two collectin-based engineered MTMs, wherein the two MTMs are selected from SEQ ID NO:6, 7, 8 or 9, or sequence variants thereof having at least 85% sequence identity to SEQ ID NO:6, 7, 8 or 9.

In a second embodiment, the invention is directed to compositions comprising ficolin-based MTMs. The ficolin-based MTMs are either naturally-occurring ficolin proteins or engineered MTM fusion proteins that comprise at least one ficolin microbe-binding domain and at least one additional domain.

The naturally-occurring ficolin protein may be any one of (i) ficolin 1, (ii) ficolin 2, (iii) ficolin 3, and (iv) a sequence variant having at least 85% sequence identity to any one of (i)-(iii).

The ficolin microbe-binding domain of the ficolin-based engineered MTMs may comprise the fibrinogen-like domain of a ficolin. The ficolin may be any one of (i) ficolin 1, (ii) ficolin 2, (iii) ficolin 3, and (iv) a sequence variant having at least 85% sequence identity to any one of (i)-(iii).

The at least one additional domain of the ficolin-based engineered MTMs may be one or more of (v) a ficolin short N-terminal domain, (vi) a ficolin collagen-like domain, (vii) a collectin cysteine-rich domain, (viii) a collectin collagen-like domain, (ix) a collectin coiled-coil neck domain, (x) a TLR transmembrane helix, (xi) a TLR C-terminal cytoplasmic signaling domain, (xii) an oligomerization domain, (xiii) a signal domain, (xiv) an anchor domain, (xv) a collagen-like domain, (xvi) a fibrinogen-like domain, (xvii) an immunoglobulin domain, (xviii) an immunoglobulin-like domain, and (xix) a sequence variant having at least 85% sequence identity to any one of (v)-(xviii).

In certain aspects of this embodiment, the at least one additional domain is an immunoglobulin domain. For example, the immunoglobulin domain may comprise the amino acid sequence of SEQ ID NO:12 or a sequence variant thereof having at least 85% sequence identity to SEQ ID NO:12.

In certain aspects of this embodiment, the ficolin-based engineered MTMs comprise a ficolin microbe-binding domain comprising the fibrinogen-like domain of any one of SEQ ID NOs: 21, 22 and 23 or a sequence variant thereof having at least 85% sequence identity to any one of SEQ ID NOs:21, 22 and 23.

In certain aspects of this embodiment, the ficolin-based engineered MTMs comprise a ficolin microbe-binding domain comprising the fibrinogen-like domain of any one of SEQ ID NOs: 21, 22 and 23 or a sequence variant thereof having at least 85% sequence identity to any one of SEQ ID NOs: 21, 22 and 23 and an immunoglobulin domain comprising the amino acid sequence of SEQ ID NO:12 or a sequence variant thereof having at least 85% sequence identity to SEQ ID NO:12.

In a third embodiment, the invention is directed to compositions comprising toll-like

receptor (TLR)-based MTMs. The TLR-based MTMs are either naturally-occurring TLR proteins or engineered MTM fusion proteins that comprise at least one TLR microbe-binding domain and at least one additional domain.

The naturally-occurring TLR protein may be any one of (i) TLR1, (ii) TLR2, (iii) TLR3, (iv) TLR4, (v) TLR5, (vi) TLR6, (vii) TLR7, (viii) TLR8, (ix) TLR9, (x) TLR10, and (xi) a sequence variant having at least 85% sequence identity to any one of (i)-(x).

The TLR microbe-binding domain of the TLR-based engineered MTMs may comprise the N-terminal ligand-binding domain of a TLR. The TLR may be any one of (i) TLR1, (ii) TLR2, (iii) TLR3, (iv) TLR4, (v) TLR5, (vi) TLR6, (vii) TLR7, (viii) TLR8, (ix) TLR9, (x) TLR10, and (xi) a sequence variant having at least 85% sequence identity to any one of (i)-(x).

The at least one additional domain of the TLR-based engineered MTMs may be one or more of (xii) a TLR transmembrane helix, (xiii) a TLR C-terminal cytoplasmic signaling domain, (xiv) a ficolin short N-terminal domain, (xv) a ficolin collagen-like domain, (xvi) a collectin cysteine-rich domain, (xvii) a collectin collagen-like domain, (xviii) a collectin coiled-coil neck domain, (xix) an oligomerization domain, (xx) a signal domain, (xxi) an anchor domain, (xxii) a collagen-like domain, (xxiii) a fibrinogen-like domain, (xxiv) an immunoglobulin domain, (xxv) an immunoglobulin-like domain, and (xxvi) a sequence variant having at least 85% sequence identity to any one of (xii)-(xxv).

In certain aspects of this embodiment, the at least one additional domain is an immunoglobulin domain. For example, the immunoglobulin domain may comprise the amino acid sequence of SEQ ID NO:12 or a sequence variant thereof having at least 85% sequence identity to SEQ ID NO:12.

In certain aspects of this embodiment, the TLR-based engineered MTMs comprise a TLR microbe-binding domain comprising the N-terminal ligand-binding domain of any one of SEQ ID NOs:24-33 or a sequence variant thereof having at least 85% sequence identity to any one of SEQ ID NOs: 24-33

In certain aspects of this embodiment, the TLR-based engineered MTMs comprise a TLR microbe-binding domain comprising the N-terminal ligand-binding domain of any one of SEQ ID NOs:24-33 or a sequence variant thereof having at least 85% sequence identity to any one of SEQ NOs:24-33 and an immunoglobulin domain comprising the amino acid sequence of SEQ ID NO:12 or a sequence variant thereof having at least 85% sequence identity to SEQ ID NO:12.

In a fourth embodiment, the invention is directed to a composition comprising one or more of the MTMs of the invention. In certain aspects of this embodiment, the composition comprises at least one naturally-occurring MTM. In certain other aspects of this embodiment, the composition comprises one collectin-based engineered MTM, wherein the collectin-based engineered MTM is an FcMBL of SEQ ID NO:6, 7, 8 or 9, or a sequence variant thereof having at least 85% sequence identity to SEQ ID NO:6, 7, 8 or 9. In certain other aspects of this embodiment, the composition comprises at least two collectin-based engineered MTMs, wherein one of the collectin-based engineered MTMs is an FcMBL of SEQ ID NO:6, 7, 8 or 9, or a sequence variant thereof having at least 85% sequence identity to SEQ ID NO:6, 7, 8 or 9. In certain other aspects of this embodiment, the composition comprises one naturally-occurring collectin MTM (e.g. MBL) and one collectin-based engineered MTM, wherein the collectin-based engineered MTM is an FcMBL of SEQ ID NO:6, 7, 8 or 9, or a sequence variant thereof having at least 85% sequence identity to SEQ ID NO:6, 7, 8 or 9. In certain other aspects of this embodiment, the composition comprises one naturally-occurring collectin MTM (e.g. MBL) and at least two collectin-based engineered MTMs, wherein one of the collectin-based engineered MTMs is an FcMBL of SEQ ID NO:6, 7, 8 or 9, or a sequence variant thereof having at least 85% sequence identity to SEQ ID NO:6, 7, 8 or 9.

In certain aspects of this embodiment, the composition further comprises a carrier or diluent, such as an aqueous solution comprising sodium acetate having a pH of about 3.2

In a fifth embodiment, the invention is directed to a composition according to any of the preceding embodiments, further comprising a therapeutic agent, for example, one or more antimicrobial agents.

In a sixth embodiment, the invention is directed to a composition according to any of the preceding embodiments, wherein at least a portion of the composition is encapsulated in at least one liposome.

In a seventh embodiment, the invention is directed to a composition according to any of the preceding embodiments, wherein the composition comprises an aerosol, a rinse, a spray, a cream, a powder, or an ointment that can be administered nasally, orally, and/or ocularly.

In an eighth embodiment, the invention is direct to a method of treating an infectious disease in a subject comprising administering a therapeutically-effective amount of a composition of the present invention to a subject having an infectious disease.

In a ninth embodiment, the invention is direct to a method of preventing an infectious disease in a subject comprising administering a therapeutically-effective amount of a composition of the present invention to a subject at risk of developing an infectious disease.

### BRIEF DESCRIPTION OF DRAWINGS

FIG. 1. Diagram showing the structure of exemplary collectins and ficolins.

### DETAILED DESCRIPTION

### I. Definitions

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

As summarized above, the present invention discloses compositions and methods for preventing and/or treating infectious disease, where the compositions comprise microbe-targeting molecules (MTMs) and/or engineered MTMs that have the shared characteristic of binding to one or more microbe-associated molecular patterns (MAMPs).

The compositions of the present invention comprise one or more MTMs, and optionally one or more therapeutic agents. A MTMs bind to, and in some cases isolates, one or more microbes or microbe components. A therapeutic agent, such as an antimicrobial agent, can optionally be included in the composition to aid in the treatment of one or more known or suspected pathogens. A composition of the present invention can comprise any suitable formulation, including, but not limited, to an aerosol, a rinse, a spray, a cream, a powder, or an ointment that can be administered nasally, orally, and/or ocularly. A method for preventing or treating an infectious disease comprises administering a therapeutically-effective amount of a composition of the invention to a subject (e.g., a human or animal subject) in need thereof. In some aspects, the formulation can be suitable for treating skin or mucosal inflammation.

The compositions and methods described herein can be used to prevent one or more pathogens from entering a subject, e.g., into the eye(s), nose, mouth, and/or respiratory system, including the airway, lungs and blood vessels, and blood; to decrease the pathogen load on the subject; and/or to treat a subject having one or more infections.

MTMs defined herein can be used to contact, and optionally isolate, microbes and microbial components based on the identity of the MAMP produced by the microbe, rather than the identity of microbe itself. While some MAMPs are produced by only a single species of microbe, other MAMPs are shared across species. Thus, while some MTMs of the invention bind to only MAMPs of a particular species of microbe, other MTMs of the invention can bind to MAMPs produced by all members of a particular class, order, family, genus or sub-genus of microbe.

As used herein, “MTM” and “engineered MTM” refers to any of the molecules described herein (or described in patents or patent application incorporated by reference) that can bind to a microbe or microbe component. The terms “microbe-targeting molecule” and “microbe-binding molecule” are used interchangeably herein.

### MAMPs

Before discussing the MTMs of the invention, it will be helpful to understand the molecules to which the MTMs bind. As indicated above, each of the MTMs of the invention binds to at least one microbe-associated molecular pattern (MAMP). Some MTMs bind at least two, at least three, at least four, at least five, or more than five MA.MPs.

As used herein and throughout the specification, the term “microbe-associated molecular patterns” or “MAMPs” refers to molecules, components or motifs associated with or secreted or released by microbes or groups of microbes (whole and/or lysed and/or disrupted) that are generally recognized by corresponding pattern recognition receptors (PRRs) of the MTM microbe-binding domains defined herein. In some aspects, the MAMPs encompass molecules associated with cellular components released during cell damage or lysis. Examples of MAMPs include, but are not limited to, microbial carbohydrates (e.g., lipopolysaccharide or LPS, mannose), endotoxins, microbial nucleic acids (e.g., bacterial, fungal or viral DNA or RNA; e.g., nucleic acids comprising a CpG site), microbial peptides (e.g., flagellin), peptidoglycans, lipoteichoic acids, N-formylmethionine, lipoproteins, lipids, phospholipids or their precursors (e.g., phosphochloline), and fungal glucans.

In some aspects, microbe components comprise cell wall or membrane components known as pathogen-associated molecular patterns (PAMPs) including lipopolysaccharide (LPS) endotoxin, lipoteichoic acid, and attached or released outer membrane vesicles. In some aspects, a microbe comprises a host cell membrane and a pathogen component or a PAMP.

In some aspects, microbe components comprise damage-associated molecular patterns (DAMPs), also known as danger-associated molecular patterns, danger signals, and alarmin. These biomolecules can initiate and sustain a non-infectious inflammatory response in a subject, in contrast to PAMPs which initiate and sustain an infectious pathogen-induced inflammatory response. Upon release from damaged or dying cells, DAMPs activate the innate immune system through binding to pattern recognition receptors (PRRs). DAMPs include portions of nuclear and cytosolic proteins, ECM (extracellular matrix), mitochondria, granules, ER (endoplasmic reticulum), and plasma membrane.

In some aspects, MAMPs include carbohydrate recognition domain (CRD)-binding motifs. As used herein, the term “carbohydrate recognition domain (CRD)-binding motifs” refers to molecules or motifs that are bound by a molecule or composition comprising a CRD (i.e. CRDs recognize and bind to CRD-binding motifs). As used herein, the term “carbohydrate recognition domain” or “CRD” refers to one or more regions, at least a portion of which, can bind to carbohydrates on a surface of microbes or pathogens. In some aspects, the CRD can be derived from a lectin, as described herein. In some aspects, the CRD can be derived from a mannose-binding lectin (MBL). Accordingly, in some aspects, MAMPs are molecules, components or motifs associated with microbes or groups of microbes that are recognized by lectin-based MTMs (collectin-based MTMs) described herein that have a CRD domain. In one embodiment, MAMPs are molecules, components, or motifs associated with microbes or groups of microbes that are recognized by mannose-binding lectin (MBL).

In some aspects, MAMPs are molecules, components or motifs associated with microbes or groups of microbes that are recognized by a C-reactive protein (CRP)-based MTMs (collectin-based MTMs).

For clarity MAMPs as used herein includes microbe components such as MAMPs, PAMPs and DAMPs as defined above.

When necessary, and unless otherwise detectable without pre-treatment, MAMPs can be exposed, released or generated from microbes in a sample by various sample pretreatment methods. In some aspects, the MAMPs can be exposed, released or generated by lysing or killing at least a portion of the microbes in the sample. Without limitations, any means known or available to the practitioner for lysing or killing microbe cells can be used. Exemplary methods for lysing or killing the cells include, but are not limited to, physical, mechanical, chemical, radiation, biological, and the like. Accordingly, pre-treatment for lysing and/or killing the microbe cells can include application of one or more of ultrasound waves, vortexing, centrifugation, vibration, magnetic field, radiation (e.g., light, UV, Vis, IR, X-ray, and the like), change in temperature, flash-freezing, change in ionic strength, change in pH, incubation with chemicals (e.g. antimicrobial agents), enzymatic degradation, and the like.

### Microbes

As used herein, the term “microbe”, and the plural “microbes”, generally refers to microorganism(s), including bacteria, virus, fungi, parasites, protozoan, archaea, protists, e.g., algae, and a combination thereof. The term “microbe” encompasses both live and dead microbes. The term “microbe” also includes pathogenic microbes or pathogens, e.g., bacteria causing diseases such as sepsis, plague, tuberculosis and anthrax; protozoa causing diseases such as malaria, sleeping sickness and toxoplasmosis; and fungi causing diseases such as ringworm, candidiasis or histoplasmosis.

In some aspects, the microbe is a human pathogen, in other words a microbe that causes at least one disease in a human.

In some aspects, the microbe is a Gram-positive bacterial species, a Gram-negative bacterial species, a mycobacterium, a fungus, a parasite, protozoa, or a virus. In some aspects, the Gram-positive bacterial species comprises bacteria from the class Bacilli. In some aspects, the Gram-negative bacterial species comprises bacteria from the class Gammaproteobacteria. In some aspects, the mycobacterium comprises bacteria from the class Actinobacteria. In some aspects, the fungus comprises fungus from the class Saccharomyces.

In some aspects, the microbe is Staphylococcus aureus, Streptococcus pyogenes, Klebsiella pneumoniae, Pseudomonas aeruginosa, Mycobacterium tuberculosis, Candida albicans, or Escherichia coli. In some aspects, the microbe is S. aureus strain 3518, S. pyogenes strain 011014, K. pneumoniae strain 631, E. coli strain 41949, P. aeruginosa strain 41504, C. albicans strain 1311, or M. tuberculosis strain H37Rv.

In some aspects, the microbe is Bartonella henselae, Borrelia burgdorferi, Campylobacter jejuni, Campylobacterfetus, Chlamydia trachomatis, Chlamydia pneumoniae, Chylamydia psittaci, Simkania negevensis, Escherichia coli (e.g., 0157:H7 and K88), Ehrlichia chafeensis, Clostridium botulinum, Clostridium perfringens, Clostridium tetani, Enterococcus faecalis, Haemophilus influenzae. Haemophilius ducreyi, Coccidioides immitis, Bordetella pertussis, Coxiella burnetii, Ureaplasma urealyticum, Mycoplasma genitalium, Trichomatis vaginalis, Helicobacter pylori, Helicobacter hepaticus, Legionella pneumophila, Mycobacterium tuberculosis, Mycobacterium bovis, Mycobacterium africanum, Mycobacterium leprae, Mycobacterium asiaticum, Mycobacterium avium, Mycobacterium celatum, Mycobacterium celonae, Mycobacterium fortuitum, Mycobacterium genavense, Mycobacterium haemophilu Mycobacterium intracellulare, Mycobacterium kansasii, Mycobacterium malmoense, Mycobacterium marinum, Mycobacterium scrofulaceum, Mycobacterium simiae, Mycobacterium szulgai, Mycobacterium ulcerans, Mycobacterium xenopi, Corynebacterium diptheriae, Rhodococcus equi, Rickettsia aeschlimannii, Rickettsia africae, Rickettsia conorii, Arcanobacterium haemolyticum, Bacillus anthracia, Bacillus cereus, Lysteria monocytogenes. Yersinia pestis, Yersinia enterocolitica, Shigella dysenteriae, Neisseria meningitides, Neisseria gonorrhoeae, Streptococcus bovis, Streptococcus hemolyticus, Streptococcus mutans, Streptococcus pyogenes, Streptococcus pneumoniae, Staphylococcus aureus, Staphylococcus epidermidis, Staphylococcus pneumoniae, Staphylococcus saprophyticus, Vibrio cholerae, Vibrio parahaemolyticus, Salmonella typhi, Salmonella paratyphi, Salmonella enteritidis, Treponema pallidum, Human rhinovirus, Human coronavirus such as SARS-COV-2, Dengue virus, Filoviruses (e.g., Marburg and Ebola viruses), Hantavirus, Rift Valley virus, Hepatitis B, C, and E, Human Immunodeficiency Virus (e.g., HIV-1, HIV-2), HHV-8, Human papillomavirus, Herpes virus (e.g., HV-I and HV-II), Human T-cell lymphotrophic viruses (e.g., HTLV-I and HTLV-II), Bovine leukemia virus, Influenza virus, Guanarito virus, Lassa virus, Measles virus, Rubella virus, Mumps virus, Chickenpox (Varicella virus), Monkey pox, Epstein Bahr virus, Norwalk (and Norwalk-like) viruses, Rotavirus, Parvovirus B19, Hantaan virus, Sin Nombre virus, Venezuelan equine encephalitis, Sabia virus, West Nile virus, Yellow Fever virus, causative agents of transmissible spongiform encephalopathies, Creutzfeldt-Jakob disease agent, variant Creutzfeldt-Jakob disease agent, Candida, Cryptcooccus, Cryptosporidium, Giardia lamblia, Microsporidia, Plasmodium vivax, Pneumocystis carinii, Toxoplasma gondii, Trichophyton mentagrophytes, Enterocytozoon bieneusi, Cyclospora cayetanensis, Encephalitozoon hellem, or Encephalitozoon cuniculi, among other viruses, bacteria, archaea, protozoa, and fungi. In yet other aspects, the microbe is a bioterror agent (e.g., B. anthracis, and smallpox).

As used herein, “microbe component” and “microbial component” refer to any part of a microbe such as cell wall components, cell membrane components, cell envelope components, cytosolic components, intracellular components, nucleic acid (DNA or RNA), or organelles in the case of eukaryotic microbes. In some aspects, the microbial component comprises a component from a Gram-positive bacterial species, a Gram-negative bacterial species, a mycobacterium, a fungus, a parasite, a virus, or any microbe described herein or known in the art.

### MTMs

As summarized above, the invention is directed to compositions and methods of using the compositions, where the compositions comprise MTMs that have the shared characteristic of binding to one or more MAMPs. The compositions of the invention can be used in the treatment and prevention of infectious diseases in a subject.

As will be apparent, while the “MTMs” of the invention include naturally-occurring molecules and proteins, the “engineered MTMs” of the invention are those have been manipulated in some manner by the hand of man. As used herein and throughout the specification, the term “engineered MTM” includes any non-naturally-occurring MTM. Engineered MTMs of the invention retain the binding specificity to a MAMP of the wild-type (i.e. naturally-occurring) molecule on which the engineered MTM is based.

The MTMs of the invention are defined based on their binding activity, therefore both naturally-occurring and engineered MTMs will comprise at least one microbe-binding domain, i.e. a domain that recognizes and binds to one or more MAMPs (including, at least two, at least three, at least four, at least five, or more) as described herein. A microbe-binding domain can be a naturally-occurring or a synthetic molecule. In some aspects, a microbe-binding domain can be a recombinant molecule.

Acceptable microbe-binding domains for use in the MTMs of the invention are limited only in their ability to recognize and bind at least one MAMP. In some aspects, the microbe-binding domain may comprise some or all of a peptide; polypeptide; protein; peptidomimetic; antibody; antibody fragment; antigen-binding fragment of an antibody; carbohydrate-binding protein; lectin; glycoprotein: glycoprotein-binding molecule; amino acid; carbohydrate (including mono-; di-; tri- and poly-saccharides); lipid; steroid; hormone; lipid-binding molecule; cofactor; nucleoside; nucleotide; nucleic acid; DNA; RNA; analogues and derivatives of nucleic acids; peptidoglycan; lipopolysaccharide; small molecule; endotoxin; bacterial lipopolysaccharide; and any combination thereof.

In particular aspects, the microbe-binding domain can be a microbe-binding domain of a lectin. An exemplary lectin is mannose binding lectin (MBL) or other mannose binding molecules. Non-limiting examples of acceptable microbe-binding domains also include microbe-binding domains from toll-like receptors, nucleotide oligomerization domain-containing (NOD) proteins, complement receptors, collectins, ficolins, pentraxins such as serum amyloid and C-reactive protein, lipid transferases, peptidoglycan recognition proteins (PGRs), and any combinations thereof. In some aspects, microbe-binding domains can be microbe-binding molecules described in the international patent application publication no. WO 2013/012924, the contents of which are incorporated by reference in their entirety.

The MTMs of the invention will typically have one or more domains in addition to a microbe-binding domain. Such domains include, but are not limited to, an oligomerization domain, a signal domain, an anchor domain, a collagen-like domain, a fibrinogen-like domain, an immunoglobulin domain, and an immunoglobulin-like domain.

Engineered MTMs of the invention include, but are not limited to, MTMs identical to a naturally-occurring MTM but having at least one amino acid change in comparison to the wild-type molecule on which they are based. Such “sequence-variant engineered MTMs” have at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity, though in all cases less than 100% sequence identity, to the wild-type molecule on which they are based. The changes may be any combination of additions, insertions, deletions and substitutions, where the altered amino acids may be naturally-occurring or non-naturally-occurring amino acids, and conservative or non-conservative changes.

Engineered MTMs of the invention also include, but are not limited to, MTMs that comprise domains from two or more different MTMs, i.e. fusion proteins. Such “domain-variant engineered MTMs” have domains from 2, 3, 4, 5 or more different proteins. For example, MTMs can be a fusion protein comprising a microbe-binding domain and an oligomerization domain, or a fusion protein comprising a microbe-binding domain and a signal domain, or a fusion protein comprising a microbe-binding domain, an oligomerization domain, and signal domain, to name a few examples. In each case, the domains within a domain-variant engineered MTM are from at least two different proteins. Other examples of such MTMs include fusion proteins comprising at least the microbe-binding domain of a lectin and at least a part of a second protein or peptide, e.g., but not limited, to an Fc portion of an immunoglobulin.

Engineered MTMs of the invention further include, but are not limited to, MTMs that comprise domains from two or more different MTMs, wherein at least one of the domains is a sequence variant of the wild-type domain upon which it is based, i.e. having at least one amino acid change in comparison to the wild-type molecule on which it is based. These “sequence- and domain-variant engineered MTMs” have domains from 2, 3, 4, 5 or more different proteins, and at least one of the domains has at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity, though in all cases less than 100% sequence identity, to the wild-type domain on which it is based. The changes may be any combination of additions, insertions, deletions and substitutions, where the altered amino acids may be naturally-occurring or non-naturally-occurring amino acids, and conservative or non-conservative changes.

As non-limiting examples of the MTMs used in the compositions and methods of the invention, three broad categories of suitable MTMs are defined in the following paragraphs, namely: (i) collectin-based MTMs, (ii) ficolin-based MTMs, and (iii) toll-like receptor-based MTMs. It should be understood that these three categories are not the only categories of MTMs encompassed by the invention.

### Collectin-based MIMs

The MTMs of the invention include collectin-based MTMs. These MTMs are either naturally-occurring collectin proteins or engineered MTM fusion proteins that comprise at least one collectin microbe-binding domain, such as the lectin carbohydrate-recognition domain (CRD), and at least one additional domain

Collectins (collagen-containing C-type lectins) are a family of collagenous calcium-dependent lectins that function in defense, thus playing an important role in the innate immune system. They are soluble molecules comprising pattern recognition receptors (PRRs) within the microbe-binding domain that recognize and bind to particular oligosaccharide structures or lipids displayed on the surface of microbes, i.e. MAMPs of oligosaccharide origin. Upon binding of collectins to a microbe, clearance of the microbe is achieved via aggregation, complement activation, opsonization, and activation of phagocytosis.

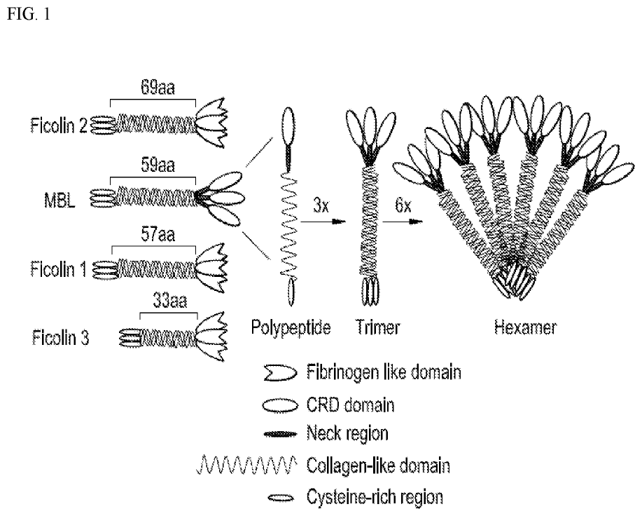

Members of the family have a common structure, characterized by four parts or domains arranged in the following N- to C-terminal arrangement: (i) a cysteine-rich domain, (ii) a collagen-like domain, (iii) a coiled-coil neck domain, and (iv) a microbe-binding domain which includes a C-type lectin domain, also termed the carbohydrate recognition domain (CRD). The functional form of the molecule is a trimer made up of three identical chains. MAMP recognition is mediated by the CRD in presence of calcium. See FIG. 1.

There are currently nine recognized members of the family: (i) mannose-binding lectin (MBL; mannose-binding lectin; e.g. SEQ ID NO:1), (ii) surfactant protein A (SP-A; SEQ ID NO:13), (iii) surfactant protein D (SP-D; SEQ ID NO:14), (iv) collectin liver 1 (CL-L1; SEQ ID NO:15), (v) collectin placenta 1 (CL-P1; SEQ ID NO:16), (vi) conglutinin collectin of 43 kDa (CL-43; SEQ ID NO:17), (vii) collectin of 46 kDa (CL-46; SEQ ID NO:18), (viii) collectin kidney 1 (CL-K1; SEQ ID NO:19), and (ix) conglutinin (SEQ ID NO:20). Each of these proteins is an MTM of the invention.

MBL (Homo sapiens; GenBank: AAH69338.1;
SEQ ID NO: 1):
MSLFPSLPLL LLSMVAASYS ETVTCEDAQK TCPAVIACSS
PGINGFPGKD GRDGTKGEKG EPGQGLRGLQ GPPGKLGPPG
NPGPSGSPGP KGQKGDPGKS PDGDSSLAAS ERKALQTEMA
RIKKWLTFSL GKQVGNKFFL INGEIMTFEK VKALCVKFQA
SVATPRNAAE NGAIQNLIKE EAFLGITDEK TEGQFVDLTG
NRLTYTNWNE GEPNNAGSDE DCVLLLKNGQ WNDVPCSTSH
LAVCEFPI
Surfactant protein A (SP-A; Homo sapiens;
GenBank: AAA36632.1; SEQ ID NO: 13):
MTFKNNTTLS LFRLENSEPL HKGYITTREL HQLALANLIF
MENCADLHFP PQMRKCFPIP GTSCLFCPKT KTLFPVLHLA
NQYLCFLCQH KHCFPQTREP DSQPSQATSR CVLSIKFTSS
IYINSHNYTL ILTCIIVRVL CPPVGCKSYK DRGHIHLAHP
SVPSTCHGSY QLTSTQYIVW ISKYDSNFQH PKLVYSPTEP
AQVEHMQCFL CMCLQREERE ALLLLPRVTI LTRLSAESTD
ERDGDSEPVN AVCRTALAFV PHESNVMLGI HNLLIWLL
Surfactant protein D (SP-D; Homo sapiens;
GenBank: AAB59450.1; SEQ ID NO: 14):
MLLELLSALV LLTQPLGYLE AEMKTYSHRT MPSACTLVMC
SSVESGLPGR DGRDGREGPR GEKGDPGLPG AAGQAGMPGQ
AGPVGPKGDN GSVGEPGPKG DTGPSGPPGP PGVPGPAGRE
GALGKQGNIG PQGKPGPKGE AGPKGEVGAP GMQGSAGARG
LAGPKGERGV PGERGVPGNT GAAGSAGAMG PQGSPGARGP
PGLKGDKGIP GDKGAKGESG LPDVASLRQQ VEALQGQVQH
LQAAFSQYKK VELFPNGQSV GEKIFKTAGF VKPFTEAQLL
CTQAGGQLAS PRSAAENAAL QQLVVAKNEA AFLSMTDSKT
EGKFTYPTGE SLVYSNWAPG EPNDDGGSED CVEIFTNGKW
NDRACGEKRL VVCEF
Collectin liver 1 (CL-L1; also known as
collectin-10; Homo sapiens; NCBI Reference
Sequence: NP_006429.2; SEQ ID NO: 15):
MNGFASLLRR NQFILLVLFL LQIQSLGLDI DSRPTAEVCA
THTISPGPKG DDGEKGDPGE EGKHGKVGRM GPKGIKGELG
DMGDQGNIGK TGPIGKKGDK GEKGLLGIPG EKGKAGTVCD
CGRYRKFVGQ LDISIARLKT SMKFVKNVIA GIRETEEKFY
YIVQEEKNYR ESLTHCRIRG GMLAMPKDEA ANTLIADYVA
KSGFFRVFIG VNDLEREGQY MFTDNTPLQN YSNWNEGEPS
DPYGHEDCVE MLSSGRWNDT ECHLTMYFVC EFIKKKK
Collectin placenta 1 (CL-P1; Homo sapiens;
GenBank: BAB72147.1; SEQ ID NO: 16):
MKDDFAEEEE VQSEGYKRFG IQEGTQCTKC KNNWALKESI
ILLYILCALL TITVAILGYK VVEKMDNVTG GMETSRQTYD
DKLTAVESDL KKLGDQTGKK AISTNSELST FRSDILDLRQ
QLREITEKTS KNKDTLEKLQ ASGDALVDRQ SQLKETLENN
SFLITTVNKT LQAYNGYVTN LQQDTSVLQG NLQNQMYSHN
VVIMNLNNLN LTQVQQRNLI TNLQRSVDDT SQAIQRIKND
FQNLQQVFLQ AKKDTDWLKE KVQSLQTLAA NNSALAKANN
DTLEDMNSQL NSFTGQMENI TTISQANEQN LKDLQDLHKD
AENRTAIKEN QLEERFQLFE TDIVNIISNI SYTAHHLRTL
TSNLNEVRTT CTDTLTKHTD DLTSLNNTLA NIRLDSVSLR
MQQDLMRSRL DTEVANLSVI MEEMKLVDSK HGQLIKNFTI
LQGPPGPRGP RGDRGSQGPP GPTGNKGQKG EKGEPGPPGP
AGERGPIGPA GPPGERGGKG SKGSQGPKGS RGSPGKPGPQ
GPSGDPGPPG PPGKEGLPGP QGPPGFQGLQ GTVGEPGVPG
PRGLPGLPGV PGMPGPKGPP GPPGPSGAVV PLALQNEPTP
APEDNGCPPH WKNFTDKCYY FSVEKEIFED AKLFCEDKSS
HLVFINTREE QQWIKKQMVG RESHWIGLTD SERENEWKWL
DGTSPDYKNW KAGQPDNWGH GHGPGEDCAG LIYAGQWNDF
QCEDVNNFIC EKDRETVLSS AL
Conglutinin collectin (CL-43; Bos taurus;
GenBank: AAI16148; SEQ ID NO: 17):
MLPLPLSILL LLTQSQSFLG EEMDVYSEKT LTDPCTVVVC
APPADSLRGH DGRDGKEGPQ GEKGDPGPPG MPGPAGREGP
SGRQGSMGPP GTPGPKGEPG PEGGVGAPGM PGSPGPTGLK
GERGTPGPGG AIGPQGPSGA MGPPGLKGDR GDPGEKGAKG
ETSVLEVDTL RQRMRNLEGE VQRLQNIVTQ YRKAVLEPDG
QAVGEKIFKT AGAVKSYSDA EQLCREAKGQ LASPRSSAEN
EAVTQLVRAK NKHAYLSMND ISKEGKFTYP TGGSLDYSNW
APGEPNNRAK DEGPENCLEI YSDGNWNDIE CREERLVICE
F
Collectin of 46 kDa (CL-46; Bos taurus;
GenBank: AAM34742; SEQ ID NO: 18):
MLLLPLSVLL LLTQPWRSLG AEMKIYSQKT LANGCTLVVC
RPPEGGLPGR DGQDGREGPQ GEKGDPGSPG PAGRAGRPGP
AGPIGPKGDN GSAGEPGPKG DTGPPGPPGM PGPAGREGPS
GKQGSMGPPG TPGPKGDTGP KGGMGAPGMQ GSPGPAGLKG
ERGAPGELGA PGSAGVAGPA GAIGPQGPSG ARGPPGLKGD
RGDPGERGAK GESGLADVNA LKQRVTILEG QLQRLQNAFS
RYKKAVLFPD GQAVGKKIFK TAGAVKSYSD AQQLCREAKG
QLASPRSAAE NEAVAQLVRA KNNDAFLSMN DISTEGKFTY
PTGESLVYSN WASGEPNNNN AGQPENCVQI YREGKWNDVP
CSEPLLVICE F
Collectin kidney 1 (CL-K1; also known as
collectin-11; Homo sapiens; GenBank:
BAF43301.1; SEQ ID NO: 19):
MRGNLALVGV LISLAFLSLL PSGHPQPAGD DACSVQILVP
GLKGDAGEKG DKGAPGRPGR VGPTGEKGDM GDKGQKGSVG
RHGKIGPIGS KGEKGDSGDI GPPGPNGEPG LPCECSQLRK
AIGEMDNQVS QLTSELKFIK NAVAGVRETE SKIYLLVKEE
KRYADAQLSC QGRGGTLSMP KDEAANGLMA AYLAQAGLAR
VFIGINDLEK EGAFVYSDHS PMRTENKWRS GEPNNAYDEE
DCVEMVASGG WNDVACHTTM YFMCEFDKEN M
Conglutinin (Bos taurus; GenBank: BAA04983;
SEQ ID NO: 20):
MLLLPLSVLL LLTQPWRSLG AEMTTFSQKI LANACTLVMC
SPLESGLPGH DGQDGRECPH GEKGDPGSPG PAGRAGRPGW
VGPIGPKGDN GFVGEPGPKG DTGPRGPPGM PGPAGREGPS
GKQGSMGPPG TPGPKGETGP KGGVGAPGIQ GFPGPSGLKG
EKGAPGETGA PGRAGVTGPS GAIGPQGPSG ARGPPGLKGD
RGDPGETGAK GESGLAEVNA LKQRVTILDG HLRRFQNAFS
QYKKAVLEPD GQAVGEKIFK TAGAVKSYSD AEQLCREAKG
QLASPRSSAE NEAVTQMVRA QEKNAYLSMN DISTEGRFTY
PTGEILVYSN WADGEPNNSD EGQPENCVEI FPDGKWNDVP
CSKQLLVICE F

The MTMs of the invention also include other collectin-based molecules that bind to one or more MAMPs, e.g. those MTMs comprising at least a portion (e.g. domain) of a lectin-based molecule in the case of an engineered MTM. As used herein, the term “collectin-based molecule” refers to a molecule comprising a microbe-binding domain derived from a collectin, such as a lectin. The term “lectin” as used herein refers to any molecule including proteins, natural or genetically modified (e.g., recombinant), that interacts specifically with saccharides (e.g., carbohydrates). The term “lectin” as used herein can also refer to lectins derived from any species, including, but not limited to, plants, animals (e.g. mammals, such as human), insects and microorganisms, having a desired carbohydrate binding specificity. Examples of plant lectins include, but are not limited to, the Leguminosae lectin family, such as ConA, soybean agglutinin, peanut lectin, lentil lectin, and Galanthus nivalis agglutinin (GNA) from the Galanthus (snowdrop) plant. Other examples of plant lectins are the Gramineae and Solanaceae families of lectins. Examples of animal lectins include, but are not limited to, any known lectin of the major groups S-type lectins, C-type lectins, P-type lectins, and I-type lectins, and galectins. In some aspects, the carbohydrate recognition domain can be derived from a C-type lectin, or a fragment thereof. C-type lectin can include any carbohydrate-binding protein that requires calcium for binding (e.g., MBL). In some aspects, the C-type lectin can include, but are not limited to, collectin, DC-SIGN, and fragments thereof. Without wishing to be bound by theory, DC-SIGN can generally bind various microbes by recognizing high-mannose-containing glycoproteins on their envelopes and/or function as a receptor for several viruses such as HIV and Hepatitis C.

Collectin-based engineered MTMs of the invention are MTMs that comprise at least a microbe-binding domain of a collectin. These MTMs may also include one or more of the other domains of a collectin, e.g. a cysteine-rich domain, a collagen-like domain, and/or a coiled-coil neck domain, as well as one or more domains not typically found in a collectin, such as an oligomerization domain, a signal domain, an anchor domain, a collagen-like domain, a fibrinogen-like domain, an immunoglobulin domain, and/or an immunoglobulin-like domain. When a collectin-based engineered MTM has each of the domains of a wild-type collectin, the MTM will be a sequence-variant engineered MTM as defined above. When a collectin-based engineered MTM has fewer that all of the domains of a wild-type collectin, the MTM will be a domain-variant engineered MTM or a sequence- and domain-variant engineered MTM as defined above.

Collectin-based engineered MTMs comprise a microbe-binding domain derived from at least one carbohydrate-binding protein selected from the group consisting of: MBL; SP-A; SP-D; CL-L1, CL-P1; CL-34; CL-46; CL-K1, conglutinin; maltose-binding protein; arabinose-binding protein; glucose-binding protein; Galanthus nivalis agglutinin; peanut lectin; lentil lectin; DC-SIGN; and C-reactive protein; and any combinations thereof.

### MBL

In particular aspects and embodiments of the invention, the MTM may be MBL, whether full-length human MBL (SEQ ID NO:1), mature human MBL without the signal sequence (e.g. SEQ ID NO:2), a truncated human MBL that retains microbe surface-binding (e.g. SEQ ID NO:3), the carbohydrate recognition domain (CRD) of human MBL (e.g. SEQ ID NO:4), or the neck and carbohydrate recognition domain of human MBL (e.g. SEQ ID NO:5), whether used alone or in combination with a second protein in the form of a fusion protein, such as a FcMBL protein as defined herein.

The amino acid sequence of full-
length human MBL (SEQ ID NO: 1;
GenBank: AAH69338.1) is:
MSLFPSLPLL LLSMVAASYS ETVTCEDAQK TCPAVIACSS
PGINGFPGKD GRDGTKGEKG EPGQGLRGLQ GPPGKLGPPG
NPGPSGSPGP KGQKGDPGKS PDGDSSLAAS ERKALQTEMA
RIKKWLTFSL GKQVGNKFFL INGEIMTFEK VKALCVKFQA
SVATPRNAAE NGAIQNLIKE EAFLGITDEK TEGQFVDLTG
NRLTYTNWNE GEPNNAGSDE DCVLLLKNGQ WNDVPCSTSH
LAVCEFPI
The amino acid sequence of mature
human MBL without the signal
sequence is:

(SEQ ID NO: 2)

ETVTCEDAQK TCPAVIACSS PGINGFPGKD GRDGTKGEKG
EPGOGLRGLQ GPPGKLGPPG NPGPSGSPGP KGOKGDPGKS
PDGDSSLAAS ERKALQTEMA RIKKWLTFSL GKQVGNKFFL
INGEIMTFEK VKALCVKFQA SVATPRNAAE NGAIQNLIKE
EAFLGITDEK TEGOFVDLTG NRLTYTNWNE GEPNNAGSDE
DCVLLLKNGQ WNDVPCSTSH LAVCEFPI
The amino acid sequence of a truncated
MBL that retains microbe surface-
binding is:

(SEQ ID NO: 3)

AASERKALQT EMARIKKWLT FSLGKQVGNK FFLINGEIMT
FEKVKALCVK FQASVATPRN AAENGAIQNL IKEEAFLGIT
DEKTEGQFVD LIGNRLTYTN WNEGEPNNAG SDEDCVLLLK
NGQWNDVPCS TSHLAVCEFP I
The amino acid sequence of the
carbohydrate recognition domain
(CRD) of human MBL is:

(SEQ ID NO: 4)

VGNKFFLING EIMTFEKVKA LCVKFQASVA TPRNAAENGA
IQNLIKEEAF LGITDEKTEG QFVDLIGNRL TYTNWNEGEP
NNAGSDEDCV LLLKNGQWND VPCSTSHLAV CEFPI
The amino acid sequence of the neck
and carbohydrate recognition domain
of MBL is:

(SEQ ID NO: 5)

PDGDSSLAAS ERKALQTEMA RIKKWLTFSL GKQVGNKFFL
TNGEIMTFEK VKALCVKFQA SVATPRNAAE NGAIQNLIKE
EAFLGITDEK TEGQFVDLTG NRLTYTNWNE GEPNNAGSDE
DCVLLLKNGQ WNDVPCSTSH LAVCEFPI

The truncated forms of the naturally-occurring protein include portions of any one of SEQ ID NOs:1-5 lacking 1, 2, 3, 4, 5, 6, 7, 8, 9, 10, 15, 20, 25, 30, 35, 40, 45, 50, 55, 60, 65, 70, 75, 80 or more amino acids from the amino-terminus of the protein, or the carboxy-terminus of the protein, or internally within the protein, or any combination thereof.

Alternatively, the truncated forms of the naturally-occurring protein of any one of SEQ ID NOs:1-5 have a deletion of at least 1%, 2%, 3%, 4%, 5%, 6%, 7%, 8%, 9%, 10%, 15%, 20%, 25%, 30%, 35%, 40%, 45% or 50% of the amino acids from the amino-terminus of the protein, or the carboxy-terminus of the protein, or internally within the protein, or any combination thereof.

As to particularly useful truncated forms of the protein, one example is the full-length amino acid sequence of the carbohydrate recognition domain (CRD) of MBL, shown in SEQ ID NO:4. In addition, suitable CRDs include CRDs having an amino acid sequence of about 10 to about 110 amino acid residues, or about 50 to about 100 amino acid residues, of SEQ ID NO:4. In some aspects, the microbe-binding domain can have an amino acid sequence of at least about 5, at least about 10, at least about 15, at least about 20, at least about 30, at least about 40, at least about 50, at least about 60, at least about 70, at least about 80, at least about 90, at least about 100, at least about 110 amino acid residues or more, of SEQ ID NO:4. Accordingly, in some aspects, the carbohydrate recognition domain of an engineered MBL protein can comprise SEQ ID NO:4. In some aspects, the carbohydrate recognition domain of an engineered MBL protein can comprise a fragment of SEQ ID NO:4 as defined above. Also, exemplary amino acid sequences of such fragments include, but are not limited to, ND, EZN (where Z is any amino acid, e.g., P), NEGEPNNAGS (SEQ ID NO:10) or a fragment thereof comprising EPN, GSDEDCVLL or a fragment thereof comprising E, and LLLKNGQWNDVPCST (SEQ ID NO:11) or a fragment thereof comprising ND. Modifications to such CRD fragments, e.g., by conservative substitution (i.e., where an amino acid is replace by an amino acid within the same class of amino acids, where the classes are: aliphatic amino acids (G, A, L, V, I); hydroxyl or sulfur/selenium-containing amino acids (S, C, U. T, M); aromatic amino acids (F, Y, W); basic amino acids (H, K, R); and acidic amino acids (D, E, N, Q)), are also within the scope described herein. In some aspects, the MBL or a fragment thereof used in the microbe surface-binding domain of the engineered MBLs described herein can be a wild-type molecule or a recombinant molecule.

The sequence variants of the naturally-occurring protein and the truncated forms thereof (e.g. SEQ ID NOs:1-5) include proteins having at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any one of SEQ ID NOs:1-5, or truncated forms thereof, that retain the ability to reduce platelet activation in blood of the protein upon which they are based.

### Engineered MTM Comprising MBL

In other particular aspects and embodiments of the invention, the MTM is an engineered MTM comprising MBL, as defined above, and a second protein in the form of a fusion protein. An exemplary fusion protein comprises some or all of naturally-occurring MBL, such as the carbohydrate recognition domain (CRD) of MBL, and a portion of an immunoglobulin, such as the Fc domain. In use, the Fc domain dimerizes and strengthens the avidity and affinity of the binding by MBLs to monomeric sugars. In some aspects, the N-terminus of fusion proteins can further comprise an oligopeptide linker adapted to bind a solid substrate and orient the CRD of the MBL domain away from a substrate to which it is immobilized. As discussed above, engineered forms of MBLs are known in the art and include each of the forms of MBL disclosed in U.S. Pat. No. 9,150,631, U.S. Patent Pub. 2016/0311877, U.S. Patent Pub. 2019/0077850, U.S. Pat. No. 9,593,160, U.S. Pat. No. 10,435,457, U.S. Patent Pub. 2015/0173883 and International Application Publication No. WO 2011/090954, the entire disclosures of which are hereby incorporated by reference in their entirety.

FcMBL is a specific engineered form of MBL of the invention that comprises the neck and CRD domains of MBL linked to an IgG Fc domain. Proline 81 of mature MBL (SEQ ID NO:2) is a convenient N-terminal point at which to begin the sequence of this engineered construct. For example, the neck and CRD domains (SEQ ID NO:5) of MBL are fused downstream (C-terminal) to the Fc domain of human IgG (Fcγ). The Fc domain may include the CH2-CH3 interface of the IgG Fc domain, which contains the binding sites for a number of Fc receptors including Staphylococcal protein A. In use, the Fc domain dimerizes and strengthens the avidity and affinity of the binding by MBLs to monomeric sugars. FcMBL is described in detail in U.S. Pat. No. 9,150,631, the entire disclosure of which is hereby incorporated by reference in its entirety.

Specific examples of FcMBLs that may be used in each of the aspects and embodiments of the invention include, but are not limited to, proteins where the neck and CRD domains of MBL are linked to an Fc component of human IgG1, with examples of the resulting constructs set forth in SEQ ID NOs:6, 7 and 9, and proteins where the CRD domain alone of MBL is linked to an Fc component of human IgG1, with an example of the resulting construct set forth in SEQ ID NO:8.

FcMBL.81 (SEQ ID NO: 6):
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GAPDGDSSLA
ASERKALQTE MARIKKWLTF SLGKQVGNKF FLINGEINIT
FEKVKALCVK FQASVATPRN AAENGAIQNL IKEEAFLGIT
DEKTEGQFVD LTGNRLTYTN WNEGEPNNAG SDEDCVLLLK
NGQWNDVPCS TSHLAVCEFP I
AKT-FcMBL (SEQ ID NO: 7):
AKTEPKSSDK THTCPPCPAP ELLGGPSVFL FPPKPKDTLM
ISRTPEVTCV VVDVSHEDPE VKENWYVDGV EVHNAKTKPR
EEQYNSTYRV VSVLTVLHQD WLNGKEYKCK VSNKALPAPI
EKTISKAKGQ PREPQVYTLP PSRDELTKNQ VSLTCLVKGF
YPSDIAVEWE SNGQPENNYK TTPPVLDSDG SFFLYSKLTV
DKSRWQQGNV FSCSVMHEAL HNHYTQKSLS LSPGAPDGDS
SLAASERKAL QTEMARIKKW LTFSLGKQVG NKFFLINGEI
MTFEKVKALC VKFQASVATP RNAAENGAIQ NLIKEEAFLG
ITDEKTEGQF VDLTGNRLTY TNWNEGEPNN AGSDEDCVLL
LKNGQWNDVP CSTSHLAVCE FPI
FcMBL.111 (SEQ ID NO: 8):
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GATSKQVGNK
FFLINGEEVI TFEKVKALCV KFQASVATPR NAAENGAIQN
LIKEEAFLGI TDEKTEGQFV DLTGNRLTYT NWNEGEPNNA
GSDEDCVLLL KNGQWNDVPC STSHLAVCEF PI
FcMBL (SEQ ID NO: 9):
AKTEPKSSDK THTCPPCPAP ELLGGPSVFL FPPKPKDTLM
ISRTPEVTCV VVDVSHEDPE VKENWYVDGV EVHNAKIKPR
EEQYDSTYRV VSVLTVLHQD WLNGKEYKCK VSNKALPAPI
EKTISKAKGQ PREPQVYTLP PSRDELTKNQ VSLTCLVKGF
YPSDIAVEWE SNGQPENNYK TIPPVLDSDG SFFLYSKLTV
DKSRWQQGNV FSCSVMHEAL HNHYTQKSLS LSPGAPDGDS
SLAASERKAL QTEMARIKKW LTESLGKQVG NKFFLINGEI
MTFEKVKALC VKFQASVATP RNAAENGAIQ NLIKEEAFLG
ITDEKTEGQF VDLTGNRLTY TNWNEGEPNN AGSDEDCVLL
LKNGQWNDVP CSTSHLAVCE FPI

In SEQ ID NO:9, the residues with a single underscore correspond to the Fc portion, the residues with a double underscore correspond to the MBL neck, and those residues without underscore correspond to the MBL carbohydrate-binding domain.

Various genetically engineered versions of the MTM (e.g., FcMBL) are described in International Application Pub. Nos. WO 2011/090954 and WO 2013/012924, as well as U.S. Pat. Nos. 9,150,631 and 9,593,160, the contents of each of which are incorporated herein by reference in their entireties. Lectins and other mannose binding molecules are also described in, for example, U.S. Pat. Nos. 9,150,631 and 9,632,085, and PCT application publication nos. WO/2011/090954, WO2013012924 and WO/2013/130875, the contents of all of which are incorporated herein by reference in their entireties.

In aspects where the MTM is FcMBL, the Fc region or a fragment thereof can comprise at least one mutation, e.g., to modify the performance of the engineered MBL. For example, in some aspects, the half-life of the engineered MBL described herein can be increased, e.g., by mutating the lysine (K) at the residue 232 to alanine (A) as shown in the Fc domain sequence provided in SEQ ID NO:12. Other mutations, e.g., located at the interface between the CH2 and CH3 domains shown in Hinton et al (2004) J Biol Chem. 279:6213-6216 and Vaccaro C. et al. (2005) Nat Biotechnol. 23: 1283-1288, can be also used to increase the half-life of the IgG1 and thus the engineered MBL.

SEQ ID NO: 12:
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GA

In some aspects and embodiments, the FcMBL of the invention comprises or consists of an amino acid sequence having at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any one of SEQ ID NOs:6-9, that retain the active of protein upon which they are based.

The exemplary MBL sequences provided herein are not construed to be limiting. For example, while the exemplary sequences provided herein are derived from a human species, amino acid sequences of the same carbohydrate recognition domain in plants and other animal species such as mice, rats, porcine, bovine, feline, and canine are known in the art and within the scope described herein.

### Other Collectins

In addition to the aspects and embodiments of the invention defined above that comprise the collectin MBL (whether naturally-occurring or engineered), the present invention encompasses use of any other collectin in an MTM that binds MAMPs, when used in conjunction with the methods and compositions of the invention. Thus, any of the following additional collectins may also be used in the aspects and embodiments of the invention as defined herein: (i) surfactant protein A (SP-A; SEQ ID NO:13), (ii) surfactant protein D (SP-D; SEQ ID NO:14), (iii) collectin liver 1 (CL-L1; SEQ ID NO:15), (iv) collectin placenta 1 (CL-P1; SEQ ID NO:16), (v) conglutinin collectin of 43 kDa (CL-43; SEQ ID NO:17), (vi) collectin of 46 kDa (CL-46; SEQ ID NO:18), (vii) collectin kidney 1 (CL-K1; SEQ ID NO:19), and (viii) conglutinin (SEQ ID NO:20).

As with MBL, both naturally-occurring collectins and engineered forms of the proteins may be used in the invention. Engineered forms of the proteins include, but are not limited to, truncated forms of the naturally-occurring proteins, sequence variants of the naturally-occurring proteins, sequence variants of the truncated forms of the proteins, fusion proteins comprising the naturally-occurring protein, fusion proteins comprising the truncated forms of the proteins, and fusion proteins comprising the sequence variants.

The truncated forms of the naturally-occurring collectins include portions of any one of SEQ ID NOs:13-20 lacking 1, 2, 3, 4, 5, 6, 7, 8, 9, 10, 15, 20, 25, 30, 35, 40, 45, 50, 55, 60, 65, 70, 75, 80 or more amino acids from the amino-terminus of the protein, or the carboxy-terminus of the protein, or internally within the protein, or any combination thereof.

Alternatively, the truncated forms of the naturally-occurring protein of any one of SEQ ID NOs:13-20 have a deletion of at least 1%, 2%, 3%, 4%, 5%, 6%, 7%, 8%, 9%, 10%, 15%, 20%, 25%, 30%, 35%, 40%, 45% or 50% of the amino acids from the amino-terminus of the protein, or the carboxy-terminus of the protein, or internally within the protein, or any combination thereof.

The sequence variants of the naturally-occurring protein and the truncated forms thereof (e.g. SEQ ID NOs:13-20) include proteins having at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any one of SEQ ID NOs: 13-20, or truncated forms thereof, that retain the activity of the protein upon which they are based.

### Ficolin-based MTMs

Ficolins are a family of lectins that activate the lectin pathway of complement activation upon binding to a pathogen. Ficolins are soluble molecules comprising pattern recognition receptors (PRRs) within a microbe-binding domain that recognize and selectively bind acetylated compounds, typically N-acetylglucosamine (GlcNAc), produced by pathogens. The lectin pathway is activated by binding of a ficolin to an acetylated compound on the pathogen surface, which activates the serine proteases MASP-1 and MASP-2, which then cleave C4 into C4a and C4b, and cleave C2 into C2a and C2b. C4b and C2b then bind together to form C3-convertase of the classical pathway, leading to the eventual lysis of the target cell via the remainder of the steps in the classical pathway.

Members of the family have a common structure, characterized by three parts or domains arranged in the following N- to C-terminal arrangement: (i) a short N-terminal domain, (ii) a collagen-like domain, and (iii) a fibrinogen-like domain that makes up the microbe-binding domain. The functional form of the molecule is a trimer made up of three identical chains. See FIG. 1.

There are currently three recognized members of the family: ficolin 1 (M-ficolin), ficolin 2 (L-ficolin), and ficolin 3 (H-ficolin). Each of these proteins is an MTM of the invention. The amino acid sequences of the human forms of the proteins are provided in the following paragraphs, with the fibrinogen-like domain underlined:

Ficolin 1 precursor - NP_001994.2 -
Homo sapiens

SEQ ID NO: 21

MELSGATMAR GLAVLLVLFL HIKNLPAQAA DTCPEVKVVG
LEGSDKLTIL RGCPGLPGAP GPKGEAGVIG ERGERGLPGA
PGKAGPVGPK GDRGEKGMRG EKGDAGQSQS CATGPRNCKD
LLDRGYFLSG WHTIYLPDCR PLTVLCDMDT DGGGWTVFQR
RMDGSVDFYR DWAAYKQGFG SQLGEFWLGN DNIHALTAQG
SSELRVDLVD FEGNHQFAKY KSEKVADEAE KYKLVLGAFV
GGSAGNSLTG HNNNFFSTKD QDNDVSSSNC AEKFQGAWWY
ADCHASNING LYLMGPHESY ANGINWSAAK GYKYSYKVSE
MKVRPA
Ficolin 2 isoform a precursor -
NP_004099.2 - Homo sapiens

SEQ ID NO: 22

MELDRAVGVL GAATLLLSFL GMAWALQAAD TCPEVKMVGL
EGSDKLTILR GCPGLPGAPG PKGEAGTNGK RGERGPPGPP
GKAGPPGPNG APGEPQPCLT GPRTCKDLLD RGHELSGWHT
IYLPDCRPLT VLCDMDTDGG GWTVFQRRVD GSVDFYRDWA
TYKQGFGSRL GEFWLGNDNI HALTAQGTSE LRVDLVDFED
NYQFAKYRSF KVADEAEKYN LVLGAFVEGS AGDSLTFHNN
QSFSTKDQDN DLNTGNCAVM FQGAWWYKNC HVSNLNGRYL
RGTHGSFANG INWKSGKGYN
Ficolin 3 isoform 1 precursor -
NP_003656.2 - Homo sapiens

SEQ ID NO: 23

MDLLWILPSL WLLLLGGPAC LKTQEHPSCP GPRELEASKV
VLLPSCPGAP GSPGEKGAPG PQGPPGPPGK MGPKGEPGDP
VNLLRCQEGP RNCRELLSQG ATLSGWYHLC LPEGRALPVF
CDMDTEGGGW LVFQRRQDGS VDFERSWSSY RAGFGNQESE
FWLGNENLHQ LTLQGNWELR VELEDENGNR TFAHYATERL
LGEVDHYQLA LGKFSEGTAG DSLSLHSGRP FTTYDADHDS
SNSNCAVIVH GAWWYASCYR SNLNGRYAVS EAAAHKYGID
WASGRGVGHP YRRVRMMLR

As with the collectins, both naturally-occurring ficolins and engineered forms of the proteins may be used in the invention. Engineered forms of the proteins include, but are not limited to, truncated forms of the naturally-occurring proteins, sequence variants of the naturally-occurring proteins, sequence variants of the truncated forms of the proteins, fusion proteins comprising the naturally-occurring protein, fusion proteins comprising the truncated forms of the proteins, and fusion proteins comprising the sequence variants.

The truncated forms of the naturally-occurring ficolins include portions of any one of SEQ ID NOs:21-23 lacking 1, 2, 3, 4, 5, 6, 7, 8, 9, 10, 15, 20, 25, 30, 35, 40, 45, 50, 55, 60, 65, 70, 75, 80 or more amino acids from the amino-terminus of the protein, or the carboxy-terminus of the protein, or internally within the protein, or any combination thereof.

Alternatively, the truncated forms of the naturally-occurring protein of any one of SEQ ID NOs:21-23 have a deletion of at least 1%, 2%, 3%, 4%, 5%, 6%, 7%, 8%, 9%, 10%, 15%, 20%, 25%, 30%, 35%, 40%, 45% or 50% of the amino acids from the amino-terminus of the protein, or the carboxy-terminus of the protein, or internally within the protein, or any combination thereof.

The sequence variants of the naturally-occurring protein and the truncated forms thereof (e.g. SEQ ID NOs:21-23) include proteins having at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any one of SEQ ID NOs:21-23, or truncated forms thereof that retain the activity of the protein upon which they are based.

The MTMs of the invention also include other ficolin-based molecules that bind to one or more MAMPs (acetylated compounds for the ficolins), e.g. those MTMs comprising at least a portion (e.g. domain) of a ficolin-based molecule in the case of an engineered MTM. As used herein, the term “ficolin-based molecule” refers to a molecule comprising a microbe-binding domain derived from a ficolin. The term “ficolin” as used herein refers to any molecule including proteins, natural or genetically modified (e.g., recombinant), that interacts specifically with acetylated compounds (e.g., GlcNAc). The term “ficolin” as used herein can also refer to ficolins derived from any species, including, but not limited to, plants, animals (e.g. mammals, such as human), insects and microorganisms, having the desired binding specificity.

Ficolin-based engineered MTMs of the invention are MTMs that comprise at least a microbe-binding domain of a ficolin, e.g. the fibrinogen-like domain of a ficolin. These MTMs may also include one or more of the other domains of a ficolin, e.g. a short N-terminal domain and/or a collagen-like domain, as well as one or more domains not typically found in a ficolin, such as a collectin cysteine-rich domain, a collectin collagen-like domain, a collectin coiled-coil neck domain, a TLR transmembrane helix, a TLR C-terminal cytoplasmic signaling domain, an oligomerization domain, a signal domain, an anchor domain, a collagen-like domain, a fibrinogen-like domain, an immunoglobulin domain, and an immunoglobulin-like domain. When a ficolin-based engineered MTM has each of the domains of a wild-type ficolin, the MTM will be a sequence-variant engineered MTM as defined above. When a ficolin-based engineered MTM has fewer that all of the domains of a wild-type ficolin, the MTM will be a domain-variant engineered MTM or a sequence- and domain-variant engineered MTM as defined above.

Ficolin-based engineered MTMs comprise a microbe-binding domain comprising at least one fibrinogen-like domain of a ficolin selected from the group consisting of ficolin 1, ficolin 2, and ficolin 3.

In certain aspects, the at least one additional domain is an immunoglobulin domain. For example, the immunoglobulin domain may comprise the amino acid sequence of SEQ ID NO:12 or a sequence variant thereof having at least 85% sequence identity to SEQ ID NO:12.

In some aspects, the MITMs and engineered MTMs of the invention comprise a microbe-binding domain comprising the fibrinogen-like domain of ficolin 1 of SEQ ID NO:21, optionally with an immunoglobulin domain of SEQ ID NO:12. In other aspects, the MTMs and engineered MTMs of the invention comprise a microbe-binding domain comprising the fibrinogen-like domain of ficolin 2 of SEQ ID NO:22, optionally with an immunoglobulin domain of SEQ ID NO:12. In further aspects, the MTMs and engineered MTMs of a microbe-binding domain comprising the fibrinogen-like domain of ficolin 3 of SEQ ID NO:23, optionally with an immunoglobulin domain of SEQ ID NO:12.

In some aspects, the engineered MTMs of the invention comprise an microbe-binding domain having an amino acid sequence selected from SEQ ID NO:21-SEQ ID NO:23, or an amino acid sequence that is at least 80, 81, 82, 83, 84, 85, 86, 87, 88, 89, 90, 91, 92, 93, 94, 95, 96, 97, 98, or 99% identical to any one of SEQ ID NO:21-SEQ ID NO:23, but less than 100% identical, and that retains the microbe-binding activity of the wild-type protein.

In some aspects, the ficolin-based engineered MTMs comprise a ficolin microbe-binding domain comprising the fibrinogen-like domain of any one of SEQ ID NOs:21, 22 and 23 or a sequence variant thereof having at least 85% sequence identity to any one of SEQ ID NOs:21, 22 and 23 and an immunoglobulin domain comprising the amino acid sequence of SEQ ID NO:12 or a sequence variant thereof having at least 85% sequence identity to SEQ ID NO:12.

In some aspects, the microbe-binding domain comprising a fibrinogen-like domain of a ficolin from a primate, mouse, rat, hamster, rabbit, or any other species as described herein.

The exemplary sequences provided herein for the ficolins are not to be construed as limiting. For example, while the exemplary sequences provided herein are derived from a human, amino acid sequences of ficolins from other species such as mice, rats, porcine, bovine, feline, and canine are known in the art and within the scope described herein.

### Toll-like Receptor-based MIMs

Toll-like receptors (TLRs) comprise a family of proteins that are integral to the proper functioning of the innate immune system. The proteins are type I integral membrane proteins (i.e. single-pass, membrane-spanning receptors) that are typically found on the surface of sentinel cells, such as macrophages and dendritic cells, but can also be found on the surface of other leukocytes including natural killer cells, T cells and B cells, and non-immune cells including epithelial cell, endothelial cells, and fibroblasts. After microbes have gained entry to a subject, such as a human, through the skin or mucosa, they are recognized by TLR-expressing cells, which leads to innate immune responses and the development of antigen-specific acquired immunity. TLRs thus recognize MAMPs by microbes.

Members of the family have a common structure, characterized by three parts or domains arranged in the following N- to C-terminal arrangement: (i) an N-terminal ligand-binding domain, i.e. the microbe-binding domain, (ii) a single transmembrane helix (˜ 20 amino acids), and (iii) a C-terminal cytoplasmic signaling domain.

The ligand-binding domain is a glycoprotein comprising 550-800 amino acid residues (depending on the identity of the TLR), constructed of tandem copies of leucine-rich repeats (LRR), which are typically 22-29 residues in length and that contains hydrophobic residues spaced at distinctive intervals. The receptors share a common structural framework in their extracellular, ligand-binding domains. The domains each adopt a horseshoe-shaped structure formed by the leucine-rich repeat motifs.

The functional form of a TLR is a dimer, with both homodimers and heterodimers being known. In the case of heterodimers, the different TLRs in the dimer may have different ligand specificities. Upon ligand binding, TLRs dimerize their ectodomains via their lateral faces, forming “m”-shaped structures. Dimerization leads to downstream signaling.

A set of endosomal TLRs comprising TLR3, TLR7, TLR8 and TLR9 recognize nucleic acids derived from viruses as well as endogenous nucleic acids in context of pathogenic events. Activation of these receptor leads to production of inflammatory cytokines as well as type I interferons (interferon type I) to help fight viral infection.

There are a number of recognized human members of the family, including TLR1, TLR2, TLR3, TLR4, TLR5, TLR6, TLR7, TLR8, TLR9, and TLR10. Each of these proteins may be used as an MTM of the invention. The amino acid sequences of the human forms of the proteins are provided in the following paragraphs, with the extracellular domain that comprises the N-terminal ligand-binding domain underlined:

TLR1 (toll-like receptor 1 precursor) - NCBI Reference
Sequence: NP 003254.2
(SEQ ID NO: 24)
MTSIFHFAII FMLILQIRIQ LSEESEFLVD RSKNGLIHVP KDLSQKTTIL NISQNYISEL
WTSDILSLSK LRILIISHNR IQYLDISVEK FNQELEYLDL SHNKLVKISC HPTVNLKHLD
LSENAFDALP ICKEFGNMSQ LKELGLSTTH LEKSSVLPIA HLNISKVLLV LGETYGEKED
PEGLQDENTE SLHIVEPINK EFHFILDVSV KTVANLELSN IKCVLEDNKC SYFLSILAKL
QTNPKLSNLT LNNIETTWNS FIRILQLVWH TTVWYFSISN VKLQGQLDER DEDYSGTSLK
ALSIHQVVSD VEGEPQSYIY EIESNMNIKN FTVSGTRMVH MLCPSKISPF LHLDESNNLL
TDTVFENCGH LTELETLILQ MNQLKELSKI AEMTTQMKSL QQLDISQNSV SYDEKKGDCS
WTKSLLSLNM SSNILTDTIF RCLPPRIKVL DLHSNKIKSI PKQVVKLEAL QELNVAENSL
TDLPGCGSFS SLSVLIIDHN SVSHPSADFF QSCQKMRSIK AGDNPFQCTC ELGEFVKNID
QVSSEVLEGW PDSYKCDYPE SYRGTLLKDF HMSELSCNIT LLIVTIVATM LVLAVTVTSL
CSYLDLPWYL RMVCQWTQTR RRARNIPLEE LQRNLQFHAF ISYSGHDSFW VKNELLPNLE
KEGMQICLHE RNFVPGKSIV ENIITCIEKS YKSIFVLSPN FVQSEWCHYE LYFAHHNLFH
EGSNSLILIL LEPIPQYSIP SSYHKLKSLM ARRTYLEWPK EKSKRGLFWA NLRAAINIKL
TEQAKK
TLR2 (toll-like receptor 2 precursor) - NCBI Reference
Sequence: NP_001305722.1
(SEQ ID NO: 25)
MPHTLWMVWV LGVIISLSKE ESSNQASLSC DRNGICKGSS GSLNSIPSGL TEAVKSLDLS
NNRITYISNS DLQRCVNLQA LVLTSNGINT IEEDSESSLG SLEHLDLSYN YLSNLSSSWF
KPLSSLTELN LLGNPYKTLG ETSLESHLTK LQILRVGNMD TFTKIQRKDF AGLTFLEELE
IDASDLQSYE PKSLKSIQNV SHLILHMKQH ILLLEIFVDV TSSVECLELR DTDLDTEHES
ELSTGETNSL IKKETERNVK ITDESLFQVM KLLNQISGLL ELEFDDCTLN GVGNFRASDN
DRVIDPGKVE TLTIRRLHIP RFYLFYDLST LYSLTERVKR ITVENSKVEL VPCLLSQHLK
SLEYLDLSEN LMVEEYLKNS ACEDAWPSLQ TLILRQNHLA SLEKTGETLL TLKNLTNIDI
SKNSFHSMPE TCQWPEKMKY LNLSSTRIHS VTGCIPKTLE ILDVSNNNLN LESLNLPQLK
ELYISRNKLM TLPDASLLPM LLVLKISRNA ITTFSKEQLD SFHTLKTLEA GGNNFICSCE
FLSFTQEQQA LAKVLIDWPA NYLCDSPSHV RGQQVQDVRL SVSECHRTAL VSGMCCALEL
LILLTGVLCH RFHGLWYMKM MWAWLQAKRK PRKAPSRNIC YDAFVSYSER DAYWVENLMV
QELENENPPF KLCLHKRDFI PGKWIIDNII DSIEKSHKTV FVLSENFVKS EWCKYELDES
HERLEDENND AAILILLEPI EKKAIPQRFC KLRKIMNTKT YLEWPMDEAQ REGEWVNLRA
AIKS
TLR3 (toll-like receptor 3 precursor) - NCBI Reference
Sequence: NP_003256.1
(SEQ ID NO: 26)
NQLRRLPAAN FTRYSQLTSL DVGENTISKL EPELCQKLPM LKVLNLQHNE LSQLSDKTFA
FCTNLTELHL MSNSIQKIKN NPFVKQKNLI TLDLSHNGLS STKLGTQVQL ENLQELLLSN
NKIQALKSEE LDIFANSSLK KLELSSNQIK EFSPGCFHAI GRLEGLELNN VQLGPSLTEK
LCLELANTSI RNLSLSNSQL STTSNTTFLG LKWTNLTMLD LSYNNLNVVG NDSFAWLPQL
EYFFLEYNNI QHLESHSLHG LENVRYLNLK RSFTKQSISL ASLPKIDDES FQWLKCLEHL
NMEDNDIPGI KSNMFTGLIN LKYLSLSNSF TSLRTLTNET FVSLAHSPLH ILNLTKNKIS
KIESDAFSWL GHLEVLDLGL NEIGQELTGQ EWRGLENIFE IYLSYNKYLQ LTRNSFALVP
SLQRLMLRRV ALKNVDSSPS PFQPLRNLTI LDLSNNNIAN INDDMLEGLE KLEILDLQHN
NLARLWKHAN PGGPIYFLKG LSHLHILNLE SNGEDEIPVE VEKDLFELKI IDLGLNNLNT
LPASVENNQV SLKSLNLQKN LITSVEKKVF GPAFRNLTEL DMRENPEDCT CESIAWFVNW
INETHTNIPE LSSHYLCNTP PHYHGFPVRL FDTSSCKDSA PFELFFMINT SILLIFIFIV
LLIHFEGWRI SFYWNVSVHR VLGFKEIDRQ TEQFEYAAYI IHAYKDKDWV WEHFSSMEKE
DQSLKFCLEE RDFEAGVFEL EAIVNSIKRS RKIIFVITHH LLKDPLCKRF KVHHAVQQAI
EQNLDSIILV FLEEIPDYKL NHALCLRRGM FKSHCILNWP VQKERIGAFR HKLQVALGSK
NSVH
TLR4 (toll-like receptor 4 isoform D) - NCBI Reference
Sequence: NP_612567.1
(SEQ ID NO: 27)
MMSASRLAGT LIPAMAFLSC VRPESWEPCV EVVPNITYQC MELNFYKIPD NLPFSTKNLD
LSFNPLRHLG SYSFFSFPEL QVLDLSRCEI QTIEDGAYQS LSHLSTLILT GNPIQSLALG
AFSGLSSLQK LVAVETNLAS LENFPIGHLK TLKELNVAHN LIQSEKLPEY ESNLTNLEHL
DLSSNKIQSI YCTDLRVLHQ MPLLNLSLDL SLNPMNFIQP GAFKEIRLHK LTLRNNEDSL
NVMKTCIQGL AGLEVHRLVL GEFRNEGNLE KEDKSALEGL CNLTIEEFRL AYLDYYLDDI
IDLFNCLTNV SSESLVSVTI ERVKDESYNF GWQHLELVNC KFGQFPTLKL KSLKRLTFTS
NKGGNAFSEV DLPSLEFLDL SRNGLSFKGC CSQSDEGTTS LKYLDLSENG VITMSSNELG
LEQLEHLDFQ HSNLKQMSEF SVELSLRNLI YLDISHTHTR VAENGIENGL SSLEVLKMAG
NSFQENFLPD IFTELRNLTF LDLSQCQLEQ LSPTAFNSLS SLQVLNMSHN NFFSLDTEPY
KCLNSLQVLD YSLNHIMTSK KQELQHFPSS LAFLNLTQND FACTCEHQSF LQWIKDQRQL
LVEVERMECA TPSDKQGMPV LSLNITCQMN KTIIGVSVLS VLVVSVVAVL VYKFYFHLML
LAGCIKYGRG ENIYDAFVIY SSQDEDWVRN ELVKNLEEGV PPFQLCLHYR DFIPGVAIAA
NIIHEGEHKS RKVIVVVSQH FIQSRWCIFE YEIAQTWQFL SSRAGIIFIV LQKVEKTLLR
QQVELYRLLS RNTYLEWEDS VLGRHIFWRR LRKALLDGKS WNPEGTVGTG CNWQEATSI
TLR5 (toll-like receptor 5 precursor) - NCBI Reference
Sequence: NP_003259.2
(SEQ ID NO: 28)
MGDHLDLLLG VVLMAGPVFG IPSCSFDGRI AFYRFCNLTQ VPQVLNTTER LLLSENYIRT
VTASSEPFLE QLQLLELGSQ YTPLTIDKEA FRNLPNLRIL DLGSSKIYFL HPDAFQGLEH
LFELRLYFCG LSDAVLKDGY FRNLKALTRL DLSKNQIRSL YLHPSEGKLN SLKSIDESSN
QIFLVCEHEL EPLQGKTLSF ESLAANSLYS RVSVDWGKCM NPERNMVLEI LDVSGNGWTV
DITGNESNAI SKSQAFSLIL AHHIMGAGFG FHNIKDPDQN TFAGLARSSV RHLDLSHGFV
FSLNSRVFET LKDLKVLNLA YNKINKIADE AFYGLDNLQV LNLSYNLIGE LYSSNFYGLP
KVAYIDLQKN HIAIIQDQTF KFLEKLQTLD LRDNALTTIH FIPSIPDIFL SGNKLVTLPK
INLTANLIHL SENRLENLDI LYELLRVPHL QILILNQNRF SSCSGDQTPS ENPSLEQLEL
GENMLQLAWE TELCWDVFEG LSHLQVLYLN HNYLNSLPPG VESHLTALRG LSLNSNRLTV
LSHNDLPANL EILDISRNQL LAPNPDVEVS LSVLDITHNK FICECELSTE INWLNHTNVT
IAGPPADIYC VYPDSESGVS LESLSTEGCD EEEVLKSLKF SLFIVCTVTL TLELMTILTV
TKFRGFCFIC YKTAQRLVFK DHPQGTEPDM YKYDAYLCFS SKDETWVQNA LLKHLDTQYS
DQNRENLCFE ERDFVPGENR IANIQDAIWN SRKIVCLVSR HELRDGWCLE AFSYAQGRCL
SDLNSALIMV VVGSLSQYQL MKHQSIRGEV QKQQYLRWPE DLQDVGWELH KLSQQILKKE
KEKKKDNNIP LQTVATIS
TLR6 (toll-like receptor 6 precursor) - NCBI Reference
Sequence: NP_006059.2
(SEQ ID NO: 29)
MTKDKEPIVK SFHFVCLMII IVGTRIQFSD GNEFAVDKSK RGLIHVPKDL PLKTKVLDMS
QNYIAELQVS DMSELSELTV LRLSHNRIQL LDLSVEKENQ DLEYLDLSHN QLQKISCHPI
VSFRHLDLSF NDFKALPICK EFGNLSQLNF LGLSAMKLQK LDLLPIAHLH LSYILLDLRN
YYIKENETES LQILNAKTLH LVFHPTSLFA IQVNISVNTL GCLQLTNIKL NDDNCQVFIK
FLSELTRGST LLNETLNHIE TTWKCLVRVF QFLWPKPVEY LNIYNLTIIE SIREEDETYS
KTTLKALTIE HITNQVFLFS QTALYTVESE MNIMMLTISD TPFIHMLCPH APSTEKELNE
TQNVFTDSIF EKCSTLVKLE TLILQKNGLK DLFKVGLMTK DMPSLEILDV SWNSLESGRH
KENCTWVESI VVLNLSSNML TDSVERCLPP RIKVLDLHSN KIKSVPKQVV KLEALQELNV
AFNSLTDLPG CGSESSLSVL IIDHNSVSHP SADFFQSCQK MRSIKAGDNP FQCTCELREF
VKNIDQVSSE VLEGWPDSYK CDYPESYRGS PLKDFHMSEL SCNITLLIVT IGATMLVLAV
TVTSLCIYLD LPWYLRMVCQ WTQTRRRARN IPLEELQRNL QFHAFISYSE HDSAWVKSEL
VPYLEKEDIQ ICLHERNEVP GKSIVENIIN CIEKSYKSIF VLSPNFVQSE WCHYELYFAH
HNLFHEGSNN LILILLEPIP QNSIPNKYHK LKALMTQRTY LQWPKEKSKR GLEWANIRAA
FNMKLTLVTE NNDVKS
TLR7 (toll-like receptor 7 precursor) - NCBI Reference
Sequence: NP_057646.1
(SEQ ID NO: 30)
GGIPTNTTNL TLTINHIPDI SPASFHRLDH LVEIDERCNC VPIPLGSKNN MCIKRLQIKP
RSFSGLTYLK SLYLDGNQLL EIPQGLPPSL QLLSLEANNI FSIRKENLTE LANIEILYLG
QNCYYRNPCY VSYSIEKDAF LNLTKLKVLS LKDNNVTAVP TVLPSTLTEL YLYNNMIAKI
QEDDENNLNQ LQILDLSGNC PRCYNAPFPC APCKNNSPLQ IPVNAFDALT ELKVLRLHSN
SLQHVPPRWF KNINKLQELD LSQNFLAKEI GDAKFLHELP SLIQLDLSEN FELQVYRASM
NLSQAFSSLK SLKILRIRGY VFKELKSENL SPLHNLQNLE VLDLGTNFIK IANLSMFKQF
KRLKVIDLSV NKISPSGDSS EVGFCSNART SVESYEPQVL EQLHYFRYDK YARSCREKNK
EASFMSVNES CYKYGQTLDL SKNSIFFVKS SDFQHLSFLK CLNLSGNLIS QTLNGSEFQP
LAELRYLDES NNRLDLLHST AFEELHKLEV LDISSNSHYF QSEGITHMLN FTKNLKVLQK
LMMNDNDISS STSRTMESES LRTLEFRGNH LDVLWREGDN RYLQLEKNLL KLEELDISKN
SLSFLPSGVF DGMPPNLKNL SLAKNGLKSF SWKKLQCLKN LETLDLSHNQ LTTVPERLSN
CSRSLKNLIL KNNQIRSLTK YFLQDAFQLR YLDLSSNKIQ MIQKTSFPEN VLNNLKMLLL
HHNRFLCTCD AVWFVWWVNH TEVTIPYLAT DVTCVGPGAH KGQSVISLDL YTCELDLTNL
ILFSLSISVS LELMVMMTAS HLYFWDVWYI YHECKAKIKG YQRLISPDCC YDAFIVYDTK
DPAVTEWVLA ELVAKLEDPR EKHENLCLEE RDWLPGQPVL ENLSQSIQLS KKTVFVMTDK
YAKTENFKIA FYLSHQRLMD EKVDVIILIF LEKPFQKSKF LQLRKRLCGS SVLEWPTNPQ
AHPYFWQCLK NALATDNHVA YSQVEKETV
TLR8 (toll-like receptor 8 isoform 1) - UniProtKB/
Swiss-Prot: Q9NR97.1
(SEQ ID NO: 31)
MENMFLQSSM LTCIFLLISG SCELCAEENF SRSYPCDEKK QNDSVIAECS NRRLQEVPQT
VGKYVTELDL SDNFITHITN ESFQGLQNLT KINLNHNPNV QHQNGNPGIQ SNGLNITDGA
FLNLKNLREL LLEDNQLPQI PSGLPESLTE LSLIQNNIYN ITKEGISRLI NLKNLYLAWN
CYFNKVCEKT NIEDGVFETL TNLELLSLSF NSLSHVPPKL PSSLRKLELS NTQIKYISEE
DEKGLINLTL LDLSGNCPRC FNAPFPCVPC DGGASINIDR FAFQNLTQLR YLNLSSTSLR
KINAAWFKNM PHLKVLDLEF NYLVGEIASG AFLTMLPRLE ILDLSFNYIK GSYPQHINIS
RNFSKLLSLR ALHLRGYVFQ ELREDDFQPL MQLPNLSTIN LGINFIKQID FKLFQNESNL
EIIYLSENRI SPLVKDTRQS YANSSSFQRH IRKRRSTDFE EDPHSNFYHF TRPLIKPQCA
AYGKALDLSL NSIFFIGPNQ FENLPDIACL NLSANSNAQV LSGTEFSAIP HVKYLDLINN
RLDFDNASAL TELSDLEVLD LSYNSHYFRI AGVTHHLEFI QNFTNLKVLN LSHNNIYTLT
DKYNLESKSL VELVESGNRL DILWNDDDNR YISIFKGLKN LTRLDLSLNR LKHIPNEAFL
NLPASLTELH INDNMLKEEN WILLQQFPRL ELLDLRGNKL LELTDSLSDF TSSLRTLLLS
HNRISHLPSG FLSEVSSLKH LDLSSNLLKT INKSALETKT TTKLSMLELH GNPFECTCDI
GDERRWMDEH LNVKIPRLVD VICASPGDQR GKSIVSLELT TCVSDVTAVI LFFFTFFITT
MVMLAALAHH LFYWDVWFIY NVCLAKVKGY RSLSTSQTFY DAYISYDTKD ASVTDWVINE
LRYHLEESRD KNVLLCLEER DWDPGLAIID NLMQSINQSK KTVFVLTKKY AKSWNFKTAF
YLALQRLMDE NMDVIIFILL EPVLQHSQYL RLRQRICKSS ILQWPDNPKA EGLEWQTLRN
VVLTENDSRY NNMYVDSIKQ Y
TLR9 (toll-like receptor 9 precursor) - NCBI Reference
Sequence: NP_059138.1
(SEQ ID NO: 32)
MGFCRSALHP LSLLVQAIML AMTLALGTLP AFLPCELQPH GLVNCNWLFL KSVPHESMAA
PRGNVTSLSL SSNRIHHLHD SDFAHLPSLR HLNLKWNCPP VGLSPMHFPC HMTIEPSTEL
AVPTLEELNL SYNNIMTVPA LPKSLISLSL SHTNILMLDS ASLAGLHALR FLEMDGNCYY
KNPCRQALEV APGALLGLGN LTHLSLKYNN LTVVPRNLPS SLEYLLLSYN RIVKLAPEDL
ANLTALRVLD VGGNCRRCDH APNPCMECPR HFPQLHPDTF SHLSRLEGLV LKDSSLSWLN
ASWFRGLGNL RVLDLSENFL YKCITKTKAF QGLTQLRKLN LSFNYQKRVS FAHLSLAPSF
GSLVALKELD MHGIFFRSLD ETTLRPLARL PMLQTLRLQM NFINQAQLGI FRAFPGLRYV
DLSDNRISGA SELTATMGEA DGGEKVWLQP GDLAPAPVDT PSSEDERPNC STLNETLDLS
RNNLVTVQPE MFAQLSHLQC LRISHNCISQ AVNGSQFLPL TGLQVLDLSH NKLDLYHEHS
FTELPRLEAL DLSYNSQPFG MQGVGHNESF VAHLRTLRHL SLAHNNIHSQ VSQQLCSTSL
RALDESGNAL GHMWAEGDLY LHFFQGLSGL IWLDLSQNRL HTLLPQTLRN LPKSLQVLRL
RDNYLAFFKW WSLHELPKLE VLDLAGNQLK ALTNGSLPAG TRLRRLDVSC NSISEVAPGE
FSKAKELREL NLSANALKTV DHSWEGPLAS ALQILDVSAN PLHCACGAAF MDELLEVQAA
VPGLPSRVKC GSPGQLQGLS IFAQDLRLCL DEALSWDCFA LSLLAVALGL GVPMLHHLCG
WDLWYCFHLC LAWLPWRGRQ SGRDEDALPY DAFVVEDKTQ SAVADWVYNE LRGQLEECRG
RWALRLCLEE RDWLPGKTLF ENLWASVYGS RKTLEVLAHT DRVSGLLRAS FLLAQQRLLE
DRKDVVVLVI LSPDGRRSRY VRLRQRLCRQ SVLLWPHQPS GQRSFWAQLG MALTRDNHHE
YNRNFCQGPT AE
TLR10 (toll-like receptor 10 isoform a) - NCBI Reference
Sequence: NP 001017388.1
(SEQ ID NO: 33)
FQLQSSDFHS VSKLRVLILC HNRIQQLDLK TFEENKELRY LDLSNNRLKS VTWYLLAGLR
YLDLSENDED TMPICEEAGN MSHLEILGLS GAKIQKSDFQ KIAHLHLNTV ELGERTLPHY
EEGSLPILNT TKLHIVLPMD TNEWVLLRDG IKTSKILEMT NIDGKSQFVS YEMQRNLSLE
NAKTSVLLLN KVDLLWDDLF LILQFVWHTS VEHFQIRNVT FGGKAYLDHN SFDYSNTVMR
TIKLEHVHER VEYIQQDKIY LLLTKMDIEN LTISNAQMPH MLFPNYPTKF QYLNFANNIL
TDELFKRTIQ LPHLKTLILN GNKLETLSLV SCFANNTPLE HLDLSQNLLQ HKNDENCSWP
ETVVNMNLSY NKLSDSVERC LPKSIQILDL NNNQIQTVPK ETIHLMALRE LNIAFNELTD
LPGCSHESRL SVLNIEMNFI LSPSLDEVQS CQEVKTLNAG RNPERCTCEL KNFIQLETYS
EVMMVGWSDS YTCEYPLNLR GTRLKDVHLH ELSCNTALLI VTIVVIMLVL GLAVAFCCLH
FDLPWYLRML GQCTQTWHRV RKTTQEQLKR NVRFHAFISY SEHDSLWVKN ELIPNLEKED
GSILICLYES YFDPGKSISE NIVSFIEKSY KSIFVLSPNF VQNEWCHYEF YFAHHNLFHE
NSDHIILILL EPIPFYCIPT RYHKLKALLE KKAYLEWPKD RRKCGLEWAN LRAAINVNVL
ATREMYELQT FTELNEESRG STISLMRTDC L

As with the collectins, both naturally-occurring TLR and engineered forms of the proteins may be used in the invention. Engineered forms of the proteins include, but are not limited to, truncated forms of the naturally-occurring proteins, sequence variants of the naturally-occurring proteins, sequence variants of the truncated forms of the proteins, fusion proteins comprising the naturally-occurring protein, fusion proteins comprising the truncated forms of the proteins, and fusion proteins comprising the sequence variants.

The truncated forms of the naturally-occurring TLRs include portions of any one of SEQ ID NOs:24-33 lacking 1, 2, 3, 4, 5, 6, 7, 8, 9, 10, 15, 20, 25, 30, 35, 40, 45, 50, 55, 60, 65, 70, 75, 80 or more amino acids from the amino-terminus of the protein, or the carboxy-terminus of the protein, or internally within the protein, or any combination thereof.

Alternatively, the truncated forms of the naturally-occurring protein of any one of SEQ ID NOs:24-33 have a deletion of at least 1%, 2%, 3%, 4%, 5%, 6%, 7%, 8%, 9%, 10%, 15%, 20%, 25%, 30%, 35%, 40%, 45% or 50% of the amino acids from the amino-terminus of the protein, or the carboxy-terminus of the protein, or internally within the protein, or any combination thereof.

The sequence variants of the naturally-occurring protein and the truncated forms thereof (e.g. SEQ ID NOs:24-33) include proteins having at least 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% sequence identity to any one of SEQ ID NOs:24-33, or truncated forms thereof that retain the activity of the protein upon which they are based.

The MTMs of the invention also include other TLR-based molecules that bind to one or more MAMPs, e.g. those MTMs comprising at least a portion (e.g. domain) of a TLR-based molecule in the case of an engineered MTM. As used herein, the term “TLR-based molecule” refers to a molecule comprising a microbe-binding domain (i.e. an N-terminal ligand-binding domain) derived from a TLR. The term “TLR” as used herein refers to any molecule including proteins, natural or genetically modified (e.g., recombinant), that interacts specifically with an MAMP and that has a Toll IL-1 receptor (TIR) domain in their signaling domain. The term “TLR” as used herein can also refer to TLR derived from any species, including, but not limited to, plants, animals (e.g. mammals, such as human), insects and microorganisms, having the desired binding specificity.

TLR-based engineered MTMs of the invention are MTMs that comprise at least a microbe-binding domain of a TLR, e.g. the N-terminal ligand-binding domain of a TLR. These MTMs may also include one or more of the other domains of a TLR, e.g. a transmembrane helix and/or a C-terminal cytoplasmic signaling domain, as well as one or more domains not typically found in a TLR, such as a ficolin short N-terminal domain, a ficolin collagen-like domain, a collectin cysteine-rich domain, a collectin collagen-like domain, a collectin coiled-coil neck domain, an oligomerization domain, a signal domain, an anchor domain, a collagen-like domain, a fibrinogen-like domain, an immunoglobulin domain, and an immunoglobulin-like domain. When a TLR-based engineered MTM has each of the domains of a wild-type TLR, the MTM will be a sequence-variant engineered MTM as defined above. When a TLR-based engineered MTM has fewer that all of the domains of a wild-type TLR, the MTM will be a domain-variant engineered MTM or a sequence- and domain-variant engineered MTM as defined above.

TLR-based engineered MTMs comprise a microbe-binding domain comprising at least one N-terminal ligand-binding domain of a TLR selected from the group consisting of TLR1, TLR2, TLR3, TLR4, TLR5, TLR6, TLR7, TLR8, TLR9, and TLR10.

In certain aspects of this embodiment, the at least one additional domain is an immunoglobulin domain. For example, the immunoglobulin domain may comprise the amino acid sequence of SEQ ID NO:12 or a sequence variant thereof having at least 85% sequence identity to SEQ ID NO:12.

In some aspects, the MITMs and engineered MTMs of the invention comprise a microbe-binding domain comprising the N-terminal ligand-binding domain of TLRI of SEQ ID NO:24, or the N-terminal ligand-binding domain of TLR2 of SEQ ID NO:25, or the N-terminal ligand-binding domain of TLR3 of SEQ ID NO:26, or the N-terminal ligand-binding domain of TLR4 of SEQ ID NO:27, or the N-terminal ligand-binding domain of TLR5 of SEQ ID NO:28, or the N-terminal ligand-binding domain of TLR6 of SEQ ID NO:29, or the N-terminal ligand-binding domain of TLR7 of SEQ ID NO:30, or the N-terminal ligand-binding domain of TLR8 of SEQ ID NO:31, or the N-terminal ligand-binding domain of TLR9 of SEQ ID NO:32, or the N-terminal ligand-binding domain of TLR10 of SEQ ID NO:33. In each of these examples, MTMs and engineered MTMs may further comprise an immunoglobulin domain of SEQ ID NO:12.

In some aspects, the engineered MTMs of the invention comprise an microbe-binding domain having an amino acid sequence selected from SEQ ID NO:24-SEQ ID NO:33, or an amino acid sequence that is at least 80, 81, 82, 83, 84, 85, 86, 87, 88, 89, 90, 91, 92, 93, 94, 95, 96, 97, 98, or 99% identical to any one of SEQ ID NO:24-SEQ ID NO:33, but less than 100% identical, and that retains the microbe-binding activity of the wild-type protein.

In certain aspects of this embodiment, the TLR-based engineered MTMs comprise a TLR microbe-binding domain comprising the N-terminal ligand-binding domain of any one of SEQ ID NOs:24-33 or a sequence variant thereof having at least 85% sequence identity to any one of SEQ ID NOs:24-33 and an immunoglobulin domain comprising the amino acid sequence of SEQ ID NO:12 or a sequence variant thereof having at least 85% sequence identity to SEQ ID NO:12.

In some aspects, the microbe-binding domain comprising a N-terminal ligand-binding domain of a TLR from a primate, mouse, rat, hamster, rabbit, or any other subject as described herein.

The exemplary sequences provided herein for the TLRs are not to be construed as limiting. For example, while the exemplary sequences provided herein are derived from a human, amino acid sequences of TLRs from other species such as mice, rats, porcine, bovine, feline, and canine are known in the art and within the scope described herein.

In some further aspects, the MTMs of the invention are those described in at least one of the following: U.S. provisional application Nos. 61/296,222, 61/508,957, 61/604,878, 61/605,052, 61/605,081, 61/788,570, 61/846,438, 61/866,843, 61/917,705, 62/201,745, 62/336,940, 62/543,614; PCT application Nos. PCT/US2011/021603, PCT/US2012/047201, PCT/US2013/028409, PCT/US2014/028683, PCT/US2014/046716, PCT/US2014/071293, PCT/US2016/045509, PCT/US2017/032928; U.S. patent application Ser. Nos. 13/574,191, 14/233,553, 14/382,043, 14/766,575, 14/831,480, 14/904,583, 15/105,298, 15/415,352, 15/483,216, 15/668,794, 15/750,788, 15/839,352, 16/059,799, 16/302,023, 16/553,635; and U.S. Pat. Nos. 9,150,631, 9,593,160, 9,632,085, 9,791,440, and 10,435,457; the contents of each of which are incorporated by reference herein in their entireties.

### Labels

The MTMs of the present invention may be labeled to allow them to be detected after binding to microbes or microbial components from a sample. The identity of the detectable label is limited only in that it can be discerned by the human eye or via a detector in the context of the detection device. Suitable detectable labels include colored or fluorescent particles, such a Europium particles or colloidal gold. Other acceptable labels include latex, which may itself be tagged with colored or fluorescent dyes, and magnetic or paramagnetic components. A further detectable label is a plasmonic fluor, wherein instead of assaying for a color change, one detects fluorescence. Ultrabright fluorescent nanolabels can also be used to improve the limit of detection in the detection devices of the invention, compared with conventional fluorophores.

Other detectable labels include, but are not limited to, an enzyme (e.g., peroxidase, alkaline phosphatase, glucose oxidase), a metal (e.g., gold for electron microscopy applications), a fluorescent marker (e.g., for immunofluorescence and flow cytometry applications, including CYE dyes, fluorescein isothiocyanate, rhodamine, phycoerytherin, phycocyanin, allophycocyanin, o-phthaldehyde and fluorescamine), a fluorescence-emitting metals (e.g., ¹⁵²Eu), a radioactive marker (e.g., radioisotopes for diagnostic purposes, including ³H, ¹³¹I, ³⁵S, ¹⁴C, and ¹²⁵I), a chemiluminescent marker (e.g., luminol, luciferin, isoluminol, theromatic acridinium ester, imidazole, acridinium salt and oxalate ester), and a protein tag (e.g., biotin, phycobiliprotein, c-Myc, HA, VSV-G, HSV, FLAG, V5, or HIS).

### Compositions Comprising MTMs

The invention also includes compositions comprising one or more of types of MTMs defined herein, i.e. both naturally-occurring MTMs and engineered MTMs. As indicated above, particular MTMs can be defined based on (i) structural terms (e.g. based on the components of the MTM; the amino acid sequence of the MTM; the nucleic acid sequence of the MTM; etc.), (ii) functional terms (e.g. the identity of the MAMP bound by the PRR portion of the microbe-binding domain; the affinity or avidity of binding to the MAMP; etc.), or (iii) both structural and functional terms. When a composition is defined as comprising two or more types of MTMs, it should be understood that “types” of MTMs in the composition differ based on structural and/or functional terms from each other. When there is more than one type of MTM in a composition, the composition is said to comprise a mixture of different types of MTMs within the composition.

The compositions may comprise different types of MTMs within one category of MTMs, as defined herein, or the compositions may comprise different types of MTMs within two or more different categories of MTMs, as defined herein. Thus, the compositions of the invention include “cocktails” of different types of MTMs, wherein the composition can include 1, 2, 3, 4, 5, 6, 7, 8, 9, 10 or more different types of MTMs within a single composition.

The compositions of the invention may comprise mixtures of naturally-occurring MTMs (e.g. MBLs), mixture of both naturally-occurring MTMs (e.g. MBLs) and the engineered MTMs defined herein (e.g. FcMBLs), or mixtures of only engineered MTMs (e.g., FcMBLs).

Depending on the manner in which the MTMs are used, the compositions comprising one or more different type of MTM may include carriers and diluents. Suitable carriers and diluents are commonly known and will vary depending on the MTM being used and the mode of use. Examples of suitable carriers and diluents include water, buffered water, saline, buffered saline, dextrose, glycerol, ethanol, and combinations thereof, propylene glycol, polysorbate 80 (Tween-80T™), poly(ethylene)glycol 300 and 400 (PEG 300 and 400), hydrophilic and hydrophobic carriers, and combinations thereof. Hydrophobic carriers include, for example, fat emulsions, lipids, PEGylated phospholipids, polymer matrices, biocompatible polymers, lipospheres, vesicles, particles, and liposomes, other stabilizing agents, solubilizing agents and surfactants, buffers and preservatives, tonicity agents, bulking agents, and lubricating agents

The compositions of the invention may also comprise one or more therapeutic agents, such as an antimicrobial agent. An MTM binds to one or more microbes or microbe components. An antimicrobial agent can optionally be included to treat (e.g. kill or inactivate) one or more known or suspected pathogens.

When the compositions comprise one or more antimicrobial agents, suitable agents include, but are not limited to, antibiotics, antivirals and antifungals. Antibiotics can be from classes including but not limited to Cephalosporin, Glycopeptide, Cyclic lipopeptide, Aminoglycoside, Macrolide, Oxazolidinone, Fluoroquinolones, Lincosamides, or Carbapenem. Antifungals can be from classes including but not limited to Polyenes, Azoles, Nucleoside Analog, Echinocandin, or Allylamine. Antivirals can be from classes including but not limited to CCR5 anatonists, Fusion inhibitors, Nucleoside/Nucleotide reverse transcriptase inhibitors (NRTIs), Non-nucleoside reverse transcriptase inhibitors (NNRTIs), Nucleotide reverse transcriptase inhibitors (NtRTIs), Integrase inhibitors, Protease inhibitors, DNA polymerase inhibitors, Guanosine analogs, Interferon-alpha, M2 ion channel blockers, Nucleoside inhibitors, NS5A polymerase inhibitors, NS3/4A protease inhibitors, Neuraminidase inhibitors, Nucleoside analogs, and Direct acting antivirals (DAAs). Examples of antimicrobials include but are not limited to aminoglycosides, ansamycins, beta-lactams, bis-biguanides, carbacephems, carbapenems, cationic polypeptides, cephalosporins, fluoroquinolones, glycopeptides, iron-sequestering glycoproteins, linosamides, lipopeptides, macrolides, monobactams, nitrofurans, oxazolidinones, penicillins, polypeptides, quaternary ammonium compounds, quinolones, silver compounds, sulfonamides, tetracyclines, and any combinations thereof.

Some exemplary antibiotics that may be included in the compositions of the invention include, but are not limited to, broad penicillins, amoxicillin (e.g., Ampicillin, Bacampicillin, Carbenicillin Indanyl, Mezlocillin, Piperacillin, Ticarcillin), Penicillins and Beta Lactamase Inhibitors (e.g., Amoxicillin-Clavulanic Acid, Ampicillin-Sulbactam, Benzylpenicillin, Cloxacillin, Dicloxacillin, Methicillin, Oxacillin, Penicillin G, Penicillin V, Piperacillin Tazobactam, Ticarcillin Clavulanic Acid, Nafcillin), Cephalosporins (e.g., Cephalosporin I Generation, Cefadroxil, Cefazolin, Cephalexin, Cephalothin, Cephapirin, Cephradine), Cephalosporin II Generation (e.g., Cefaclor, Cefamandole, Cefonicid, Cefotetan, Cefoxitin, Cefprozil, Cefmetazole, Cefuroxime, Loracarbef), Cephalosporin III Generation (e.g., Cefdinir, Ceftibuten, Cefoperazone, Cefixime, Cefotaxime, Cefpodoxime proxetil, Ceftazidime, Ceftizoxime, Ceftriaxone), Cephalosporin IV Generation (e.g., Cefepime), Macrolides and Lincosamides (e.g., Azithromycin, Clarithromycin, Clindamycin, Dirithromycin, Erythromycin, Lincomycin, Troleandomycin), Quinolones and Fluoroquinolones (e.g., Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, Perfloxacin), Carbapenems (e.g., Imipenem-Cilastatin, Meropenem), Monobactams (e.g., Aztreonam), Aminoglycosides (e.g., Amikacin, Gentamicin, Kanamycin, Neomycin, Netilmicin, Streptomycin, Tobramycin, Paromomycin), Glycopeptides (e.g., Teicoplanin, Vancomycin), Tetracyclines (e.g., Demeclocycline, Doxycycline, Methacycline, Minocycline, Oxytetracycline, Tetracycline, Chlortetracycline), Sulfonamides (e.g., Mafenide, Silver Sulfadiazine, Sulfacetamide, Sulfadiazine, Sulfamethoxazole, Sulfasalazine, Sulfisoxazole, Trimethoprim-Sulfamethoxazole, Sulfamethizole), Rifampin (e.g., Rifabutin, Rifampin, Rifapentine), Oxazolidinones (e.g., Linezolid, Streptogramins, Quinupristin Dalfopristin), Bacitracin, Chloramphenicol, Fosfomycin, Isoniazid, Methenamine, Metronidazole, Mupirocin, Nitrofurantoin, Nitrofurazone, Novobiocin, Polymyxin, Spectinomycin, Trimethoprim, Colistin, Cycloserine, Capreomycin, Ethionamide, Pyrazinamide, Para-aminosalicylic acid, Erythromycin ethylsuccinate, and the like.

Some exemplary antifungals that may be included in the compositions of the invention include, but are not limited to, polyene antifungals, Amphotericin B, Candicidin, Filipin, Hamycin, Natamycin, Nystatin, Rimocidin, imidazole antifungals, triazole antifungals, thiazole antifungals, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Triazoles[edit], Albaconazole, Efinaconazole, Epoxiconazole, Fluconazole, Isavuconazole, Itraconazole, Posaconazole, Propiconazole, Ravuconazole, Terconazole, Voriconazole, Abafungin, Allylamines, amorolfin, butenafine, naftifine, terbinafine, Echinocandins, Anidulafungin, Caspofungin, Micafungin, Aurones, Benzoic acid, Ciclopirox, Flucytosine, 5-fluorocytosin, Griseofulvin, Haloprogin, Tolnaftate, Undecylenic acid, Triacetin, Crystal violet, Castellani's paint, Orotomide, Miltefosine, Potassium iodide, Coal tar, Copper(II) sulfate, Selenium disulfide, Sodium thiosulfate, Piroctone olamine, Iodoquinol, clioquinol, Acrisorcin, Zinc pyrithione, and Sulfur. Additional antifungals known in the art can also be used.

Some exemplary antivirals that may be included in the compositions of the invention include, but are not limited to, Abacavir, Acyclovir, Adefovir, Amantadine, Ampligen, Amprenavir, antiretroviral, Arbidol, Atazanavir, Atripla, Boceprevir, Cidofovir, Combivir, Daclatasvir, Darunavir, Delavirdine, Dasabuvir, Didanosine, Docosanol, Dolutegravir, Doravirine, Ecoliever, Edoxudine, Efavirenz, Elbasvir, Emtricitabine, Enfuvirtide, Entecavir, Etravirine, Famciclovir, Fomivirsen, Fosamprenavir, Foscarnet, Fosfonet, Fusion inhibitor, Ganciclovir, Gemcitabine, Glecaprevir, Grazoprevir, Ibacitabine, Idoxuridine, Imiquimod, Imunovir, Indinavir, Inosine, Integrase inhibitor, Interferon, Interferon type I, Interferon type II, Interferon type III, Lamivudine, Ledipasvir, Lopinavir, Lopiravir, Loviride, Maraviroc, Methisazone, Moroxydine, Nelfinavir, Nevirapine, Nexavir, Nitazoxanide, Norvir, Nucleoside analogues, Ombitasvir, Oseltamivir (Tamiflu), Paritaprevir, Peglyated Interferon-alpha, Peginterferon alfa-2a, Penciclovir, Peramivir, Pibrentasvir, Pleconaril, Podophyllotoxin, Protease inhibitor, Pyramidine, Raltegravir, Reverse transcriptase inhibitor, Ribavirin, Rilpivirine, Rimantadine, Ritonavir, Saquinavir, Simeprevir, Sofosbuvir, Stavudine, Synergistic enhancer (antiretroviral), Telaprevir, Telbivudine, Tenofovir, Tenofovir disoproxil, Tipranavir, Trifluridine, Trizivir, Tromantadine, Truvada, Valaciclovir (Valtrex), Valganciclovir, Velpatasvir, Vicriviroc, Vidarabine, Viramidine, Voxilaprevir, Zalcitabine, Zanamivir (Relenza), Zidovudine. Additional antivirals known in the art can also be used.

The compositions of the invention can take many different forms, varying widely based on (i) the identity of the MTMs in the composition, (ii) the identity of other components in the composition, and (iii) the intended use of the composition, to name only a few of the relevant factors.

For pharmaceutical uses, the compositions of the invention may further comprise pharmaceutically acceptable carriers and diluents when administered to or used on a living subject. Suitable carriers and diluents are commonly known and will vary depending on the MTM being used or administered and the mode of use or administration. Examples of suitable carriers and diluents include saline, buffered saline, dextrose, water-for-injection, glycerol, ethanol, and combinations thereof, propylene glycol, polysorbate 80 (Tween-80™), poly(ethylene)glycol 300 and 400 (PEG 300 and 400), PEGylated castor oil (e.g. Cremophor EL), poloxamer 407 and 188, a cyclodextrin or a cyclodextrin derivative (including HPCD ((2-hydroxypropyl)-cyclodextrin) and (2-hydroxyethyl)-cyclodextrin), hydrophilic and hydrophobic carriers, and combinations thereof. Hydrophobic carriers include, for example, fat emulsions, lipids, PEGylated phospholipids, polymer matrices, biocompatible polymers, lipospheres, vesicles, particles, and liposomes, other stabilizing agents, solubilizing agents and surfactants, buffers and preservatives, tonicity agents, bulking agents, and lubricating agents. The formulations comprising MTMs will typically have been prepared using MTMs proteins from cultures prepared in the absence of any non-human components, such as animal serum (e.g., bovine serum albumin).

As described above, a composition of the present invention can comprise any suitable formulation, including but not limited to an aerosol, a rinse, a spray, a cream, a powder, or an ointment that can be administered nasally, orally, and/or ocularly.

In some aspects, a composition of the present invention comprises an aerosolized composition. The composition can comprise a solution, e.g., a liquid, of MTMs that can be aerosolized by any aerosol generating/delivery device as known in the art (described below). The aerosolized particles can be of a suitable size for deposition into the lungs, including the smaller airways and alveoli, or onto a carrier or inert surface. An exemplary sized particle can range from about 1 micron to about 5 microns, for example, about 1 micron, or about 2 microns, or about 3 microns, or about 4 microns, or about 5 microns.

An aerosolized composition can be delivered to a subject, e.g., via the nose, eyes or mouth, by any aerosol generating/delivery device as known in the art. In some aspects, the delivery device comprises a nebulizer where the nebulizer can comprise a small volume nebulizer, a large volume nebulizer, or an ultrasound nebulizer. In some aspects, the delivery device comprises a metered-dose inhaler. In these aspects, the inhaler can include spacers or holding chambers. In some aspects, the delivery device comprises a dry-powder inhaler.

The aerosolized composition can be administered to a subject preventatively to prevent one or more pathogens from entering a subject, e.g., into the eye(s), nose, mouth, and/or respiratory system including the airway, lungs and blood vessels, and blood. The MTMs of the composition act by binding to one or more microbes or microbe components and preventing the one or more microbe or microbe components, thus immobilizing it and preventing it from entering the eye(s), nose, mouth, and/or respiratory system including the airway, lungs and blood vessels, and ultimately the blood. Aerosol administration enables a direct, topical application of the composition to the target site, such as the lung, which may be particularly advantageous in preventing a respiratory-transmitted infection such as SARS-COV-2.

In some aspects, a composition of the present invention comprises an ocular rinse or ointment. The composition can be mixed with any suitable biocompatible ocular rinses that are known in the art. The composition can be administered to a subject by dropping the rinse into the eye, or applying an ointment proximate to the eye, e.g., the eye-lids.

In some aspects, a composition of the present invention comprises a mouth rinse. The composition can be mixed with any suitable biocompatible mouth rinses that are known in the art. The composition can be administered to a subject's mouth by swooshing, gargling and/or swallowing the rinse.

In some aspects, a composition of the present invention comprises a nasal rinse, spray or ointment. The composition can be mixed with any suitable biocompatible nasal rinses or spray that are known in the art. The composition can be administered to a subject by dropping or spraying into the nose, or applying an ointment in or proximate to the nose, e.g., the nostrils, to prevent pathogens from binding to the nasal mucosa. For example, the compositions of the present invention can be used to block binding of SARS-COV-2 to ACE2 receptors in the nasal cavity.

In some aspects, the composition can be delivered via any suitable delivery device. In some aspects, the delivery device can be configured to deliver liquids and powders formulations. In some aspects, the delivery device can be configured to deliver liquid formulations, for example, via a pipette, a rhinyle catheter and squirt tube, a squeeze bottle, a metered dose spray pump, a single or duo-dose spray device, a nasal pressurized metered-dose inhaler, or a device comprising a suitable geometry for targeting anterior nose as it relate to the spray plume. In some aspects, the delivery device can be configured to deliver powder formulations, for example, via a nasal powder inhaler, a nasal powder sprayer, a nasal powder insufflator, or a breath powered bi-directional device.

As indicated above, the compositions can comprise a carrier. In some aspects, the carrier is a liposome, where the MTM is encapsulated in one or more liposomes. Encapsulation of the MTMs may provide certain characteristics, including but not limited to, altered drug pharmacokinetics and biodistribution, sustained release from the liposome, enhanced delivery to target sites, and protection of the composition from degradation. Any suitable liposome composition and encapsulation method known in the art may be used.

In some aspects, a portion of the composition can be encapsulated and a portion is not encapsulated, for example to provide a high therapeutic level of treatment to the site, while also maintaining a sustained release of the composition over time.

In some aspects, the composition comprises an aqueous solution. In some aspects, the composition comprises sodium acetate.

In some aspects, the composition comprises a pH ranging from 3 to 7.6.

In some aspects, the composition comprises an MTM concentration ranging between about 0.01% and about 20%, such as between about 0.1% and about 10%, such as between about 0.5% and about 5%.

In some aspects, the composition comprises at least one antimicrobial agent, DC-SIGN, and a ficolin.

In some aspects, the composition comprises one or more MTMs, encapsulated or unencapsulated, or a mixture thereof, at a concentration from about 1% to about 99%, for example, greater than about 10%, greater than about 20%, greater than about 30%, greater than about 40%, greater than about 50%, greater than about 60%, greater than about 70%, greater than about 80%, such as about 81%, such as about 82%, such as about 83%, such as about 84%, such as about 85%, such as about 86%, such as about 87%, such as about 88%, such as about 89%, such as about 90%, such as about 91%, such as about 92%, such as about 93%, such as about 94%, such as about 95%, such as about 96%, such as about 97%, such as about 98%, such as about 99%, such as about 100%.

In some aspects, the composition comprises an MTM homopolymer. In some aspects, the composition comprises an MTM heterpolymer.

In any of the above aspects, the composition can be sterile.

In a nonlimiting example, the composition can comprise unencapsulated MTM and encapsulated MTM, where the MTM comprises FcMBL, in an aqueous solution comprising sodium acetate having a pH of about 3.2, where the composition is formulated for aerosolized delivery to animal or human nasal and oral cavities, where the particles of the aerosol comprise a diameter from about 2 microns to about 4 microns, and where the unencapsulated MTM is in the aqueous solution at a concentration of about 30 mg/mL.

In a nonlimiting example, the composition can comprise unencapsulated MTM and encapsulated MTM, where the MTM comprises a lectin, in an aqueous solution comprising sodium acetate having a pH of about 3.2, where the composition is formulated for aerosolized delivery to animal or human nasal and oral cavities, where the particles of the aerosol comprise a diameter from about 2 microns to about 4 microns, and where the unencapsulated MTM is in the aqueous solution at a concentration of about 10 mg/mL.

In a nonlimiting example, the composition can comprise unencapsulated MTM and encapsulated MTM, where the encapsulated MTM comprises truncated MBL, in an aqueous solution comprising sodium acetate having a pH of about 3.2, where the composition is formulated for aerosolized delivery to animal or human nasal and oral cavities, where the particles of the aerosol comprise a diameter from about 1 microns to about 5 microns.

Any of the compositions described herein can be administered to a subject after exposure to a pathogen, for example, to decrease the pathogen load on the subject, to treat a subject having one or more infections, and to prevent the spread of pathogens to others. In some aspects, if a subject is exposed to a known pathogen, a particular antimicrobial, based on the pathogen, can be selected and added to the composition.

### Methods

The invention includes methods of treating infectious disease in a subject. Exemplary methods include a method of treating an infectious disease in a subject comprising administering a therapeutically-effective amount of a composition of the present invention to a subject having an infectious disease. The therapeutically-effective amount will be sufficient to decrease or lessen at least one symptom of the infectious disease.

The invention includes methods of preventing infectious disease in a subject. Exemplary methods include a method of preventing an infectious disease in a subject comprising administering a therapeutically-effective amount of a composition of the present invention to a subject at risk of developing an infectious disease. Such preventing can be used to block entry of the microbe into the body of the subject and/or block development of disease in the subject when the microbe has already entered the body of the subject. In this context, blocking entry should be understood to mean passage of the microbe through the skin, tissue (e.g. lung tissue or tissue of the nose), conjunctiva (e.g. eye tissue) or other barrier between the cells of the body and the environment. The therapeutically-effective amount will be sufficient to decrease or lessen the amount of microbe entering the body, or the amount sufficient to (i) decrease or lessen the numbers of microbe in the body and/or (ii) decrease or lessen growth of the microbe in the body.

### Subject

As used herein, a “subject” may be human, a non-human primate, bird, horse, cow, goat, sheep, a companion animal, such as a dog, cat or rodent, or other mammal.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

In some aspects, compositions, methods, systems, and assays are further described in at least one of the following: U.S. provisional application numbers 61/296,222, 61/508,957, 61/604,878, 61/605,052, 61/605,081, 61/788,570, 61/846,438, 61/866,843, 61/917,705, 62/201,745, 62/336,940, 62/543,614; PCT application numbers PCT/US2011/021603, PCT/US2012/047201, PCT/US2013/028409, PCT/US2014/028683, PCT/US2014/046716, PCT/US2014/071293, PCT/US2016/045509, PCT/US2017/032928; U.S. patent application Ser. Nos. 13/574,191, 14/233,553, 14/382,043, 14/766,575, 14/831,480, 14/904,583, 15/105,298, 15/415,352, 15/483,216, 15/668,794, 15/750,788, 15/839,352, 16/059,799, 16/302,023, 16/553,635; and U.S. Pat. Nos. 9,150,631, 9,593,160, 9,632,085, 9,791,440, and 10,435,457; the contents of each of which are incorporated by reference herein in their entireties.

### EQUIVALENTS

Various modifications of the invention and many further aspects thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various aspects and equivalents thereof.

What is claimed is:

1. A composition comprising a collectin-based engineered microbe-targeting molecule (MTM) comprising at least one collectin microbe-binding domain and at least one additional domain, wherein
the collectin microbe-binding domain comprises the carbohydrate recognition domain (CRD) of a collectin selected from the group consisting of
(i) mannose-binding lectin (MBL),
(ii) surfactant protein A (SP-A),
(iii) surfactant protein D (SP-D),
(iv) collectin liver 1 (CL-L1),
(v) collectin placenta 1 (CL-P1),
(vi) conglutinin collectin of 43 kDa (CL-43),
(vii) collectin of 46 kDa (CL-46),
(viii) collectin kidney 1 (CL-K1),
(ix) conglutinin, and
(x) a sequence variant having at least 85% sequence identity to any one of (i)-(ix), and wherein the at least one additional domain is one or more domains selected from the group consisting of
(xi) a collectin cysteine-rich domain,
(xii) a collectin collagen-like domain,
(xiii) a collectin coiled-coil neck domain,
(xiv) a ficolin short N-terminal domain,
(xv) a ficolin collagen-like domain,
(xvi) a TLR transmembrane helix,
(xvii) a TLR C-terminal cytoplasmic signaling domain,
(xviii) an oligomerization domain,
(xix) a signal domain,
(xx) an anchor domain,
(xxi) a collagen-like domain,
(xxii) a fibrinogen-like domain,
(xxiii) an immunoglobulin domain,
(xxiv) an immunoglobulin-like domain, and
(xxv) a sequence variant having at least 85% sequence identity to any one of (xi)-(xxiv).

2. The composition of claim 1, wherein the collectin microbe-binding domain comprises the CRD of MBL or a sequence variant thereof having at least 85% sequence identity to the CRD of MBL.

3. The composition of claim 1 or 2, wherein the CRD of MBL comprises the amino acid sequence of any one of SEQ ID NOs:1, 2, 3, 4, and 5 or a sequence variant thereof having at least 85% sequence identity to any one of SEQ ID NOs:1, 2, 3, 4, and 5.

4. The composition of claim 1, wherein the at least one additional domain is an immunoglobulin domain.

5. The composition of claim 1 or 4, wherein the immunoglobulin domain comprises the amino acid sequence of SEQ ID NO:12 or a sequence variant thereof having at least 85% sequence identity to SEQ ID NO:12.

6. The composition of claim 1 or 4, wherein the MTM is one or more of SEQ ID NOs: 6-9, or a sequence variant thereof having at least 85% sequence identity to any one of SEQ ID NOs:6-9.

7. A composition comprising a ficolin-based engineered microbe-targeting molecule (MTM) comprising at least one ficolin microbe-binding domain and at least one additional domain, wherein
the ficolin microbe-binding domain comprises the fibrinogen-like domain of a ficolin selected from the group consisting of
(i) ficolin 1,
(ii) ficolin 2,
(iii) ficolin 3, and
(iv) a sequence variant having at least 85% sequence identity to any one of (i)-(iii), and wherein the at least one additional domain is one or more domains selected from the group consisting of
(v) a ficolin short N-terminal domain,
(vi) a ficolin collagen-like domain,
(vii) a collectin cysteine-rich domain,
(viii) a collectin collagen-like domain,
(ix) a collectin coiled-coil neck domain,
(x) a TLR transmembrane helix,
(xi) a TLR C-terminal cytoplasmic signaling domain,
(xii) an oligomerization domain,
(xiii) a signal domain,
(xiv) an anchor domain,
(xv) a collagen-like domain,
(xvi) a fibrinogen-like domain,
(xvii) an immunoglobulin domain,
(xviii) an immunoglobulin-like domain, and
(xix) a sequence variant having at least 85% sequence identity to any one of (v)-(xviii).

8. The composition of claim 7, wherein the ficolin microbe-binding domain comprises the fibrinogen-like domain of any one of SEQ ID NOs:21, 22 and 23, or a sequence variant thereof having at least 85% sequence identity to any one of SEQ ID NOs:21, 22 and 23.

9. The composition of claim 7 or 8, wherein the at least one additional domain is an immunoglobulin domain.

10. The composition of any one of claims 7-9, wherein the immunoglobulin domain comprises the amino acid sequence of SEQ ID NO:12 or a sequence variant thereof having at least 85% sequence identity to SEQ ID NO:12.

11. A composition comprising a toll-like receptor (TLR)-based engineered microbe-targeting molecule (MTM) comprising at least one TLR microbe-binding domain and at least one additional domain, wherein
the TLR microbe-binding domain comprises the N-terminal ligand-binding domain of a TLR selected from the group consisting of
(i) TLR1,
(ii) TLR2,
(iii) TLR3,
(iv) TLR4,
(v) TLR5,
(vi) TLR6,
(vii) TLR7,
(vii) TLR8,
(viii) TLR9,
(ix) TLR10, and
(x) a sequence variant having at least 85% sequence identity to any one of (i)-(ix), and wherein the at least one additional domain is one or more domains selected from the group consisting of
(xi) a TLR transmembrane helix,
(xii) a TLR C-terminal cytoplasmic signaling domain,
(xiii) a ficolin short N-terminal domain,
(xiv) a ficolin collagen-like domain,
(xv) a collectin cysteine-rich domain,
(xvi) a collectin collagen-like domain,
(xvii) a collectin coiled-coil neck domain,
(xviii) an oligomerization domain,
(xix) a signal domain,
(xx) an anchor domain,
(xxi) a collagen-like domain,
(xxii) a fibrinogen-like domain,
(xxiii) an immunoglobulin domain,
(xxiv) an immunoglobulin-like domain, and
(xxv) a sequence variant having at least 85% sequence identity to any one of (xi)-(xxiv).

12. The composition of claim 11, wherein the TLR microbe-binding domain comprises the N-terminal ligand-binding domain of any one of SEQ ID NOs:24-33 or a sequence variant thereof having at least 85% sequence identity to any one of SEQ ID NOs:24-33.

13. The composition of claim 11 or 12, wherein the at least one additional domain is an immunoglobulin domain.

14. The composition of any one of claims 11-13, wherein the immunoglobulin domain comprises the amino acid sequence of SEQ ID NO:12 or a sequence variant thereof having at least 85% sequence identity to SEQ ID NO:12.

15. A composition comprising one or more engineered MTMs, wherein the one or more engineered MTMs is selected from any of the engineered MTMs of claims 1-14.

16. The composition of claim 15, further comprising at least one naturally-occurring MTM.

17. The composition of claim 15, further comprising one or more antimicrobial agents.

18. The composition of claim 16, further comprising one or more antimicrobial agents.

19. The composition of claim 15, comprising at least two collectin-based engineered MTMs of claim 1, wherein at least one of the collectin-based engineered MTMs is an FcMBL of SEQ ID NO:6, 7, 8 or 9 or a sequence variant thereof having at least 85% sequence identity to SEQ ID NO:6, 7, 8 or 9.

20. The composition according to any preceding claim, wherein at least a portion of the composition is encapsulated in at least one liposome.

21. The composition according to any preceding claim, wherein the composition comprises an aerosol, a rinse, a spray, a cream, a powder, or an ointment formulated for nasal, oral, and/or ocular administration.

22. A method of treating an infectious disease in a subject comprising administering a therapeutically-effective amount of a composition of any preceding claim to a subject having an infectious disease.

23. A method of preventing an infectious disease in a subject comprising administering a therapeutically-effective amount of a composition of any preceding claim to a subject at risk of developing an infectious disease.
